(19) United States
(12) Patent Application Publication
(10) Pub. No.: US 2023/0414811 A1
(43) Pub. Date: Dec. 28, 2023

(54) LIQUID PROCESSING APPARATUS WITH ATMOSPHERIC, LOW-TEMPERATURE PLASMA ACTIVATION

(71) Applicant: TellaPure, LLC, Reston, VA (US)

(72) Inventors: Richard P. Lawless, Great Falls, VA (US); Edgar Bryan Hill, Ashburn, VA (US); Dawn Price, Great Falls, VA (US); Hidetaka Noda, Tokyo (JP); Akitoshi Okino, Tokyo (JP)

(73) Assignee: TellaPure, LLC, Reston, VA (US)

(21) Appl. No.: 17/932,059

(22) Filed: Sep. 14, 2022

Related U.S. Application Data
(63) Continuation of application No. 17/868,730, filed on Jul. 19, 2022, now abandoned.
(60) Provisional application No. 63/355,633, filed on Jun. 26, 2022.

(51) Int. Cl. A61L 2/14; A61B 18/04

(52) U.S. Cl. CPC A61L 2/14; A61B 18/042

(57) ABSTRACT

A plasma generator apparatus generates atmospheric pressure and low temperature plasma that can communicate with a gas to generate reactive species that can be contained in a liquid. The plasma generator has a first electrode and a second electrode opposing each other with a space there between that is configured to house the gas. The generator also has a dielectric layer having a relative permittivity between 2 and 500, and a thickness of 3 mm or less; a power supply configured to supply electrical power to the first and second electrodes at a predetermined voltage and frequency, such that, based on a distance between the first and second electrodes, and the presence of the dielectric layer, atmospheric pressure, low temperature plasma is generated in the space so as to communicate with the gas disposed therein to thereby generate the reactive species.

### BACKGROUND

Some embodiments relate to methods and apparatus for generating, dispersing, ejecting, controlling, and/or using free radicals. Some of these embodiments more specifically relate to methods and apparatus for generating the free radicals via atmospheric pressure, low-temperature plasma (hereinafter “cold plasma” or “non-equilibrium plasma” or “stable plasma” or “plasma”). Some embodiments use these free radicals to affect or breakdown molecules in order to neutralize harmful matter dissolved in fluids or on the surfaces or pores of solid objects.

Cold plasma is plasma where the temperatures of the individual constituents are different from each other. Electrons exist at higher temperatures (more than 10,000K) and neutral atoms can exist at room temperature. However, the density of the electrons in cold plasma is very low compared to the density of the neutral atoms. In a laboratory, cold plasmas are generally produced by applying electrical energy to different inert gases. This production can be performed at room temperature and at atmospheric pressure, obviating costly instruments and thereby reducing the overall costs associated with making cold plasma.

On a molecular level, cold plasma can be produced by moving accelerated electrons through certain gasses, e.g., helium or air. These electrons impact the atoms and molecules with so much energy that they separate the outermost electrons of the atoms and molecules in the gas, thereby creating a soupy mixture of free electrons and free ions. The gas remains at approximately room temperature because the energy required to separate the electrons from their atoms quickly dissipates, leaving the gas ions cool. The relatively low-density plasma enables controlled ionization of the available gas, which generates little to no detectable sound.

On a practical level, cold plasma is produced based on dielectric-barrier discharge (DBD) technology, which is the electrical discharge between two electrodes separated by one or more insulating dielectric barriers. DBD has been referred to as silent (inaudible) discharge, ozone production discharge, or partial discharge. In the related art, DBD requires high voltage alternating current ranging from lower radio frequency to microwave frequencies. Plasma is produced by two electrodes with a dielectric layer between the electrodes to limit the current flow in the plasma. The dielectric layer that limits the current controls the rate of ionization of the gas. DBD constitutes a dry method of plasma production that does not generate wastewater or require drying of the material after treatment.

### SUMMARY

Creating stable cold plasma can be difficult because it involves balancing numerous factors. For example, changes in voltage, composition of gases entering the system, air flow rate, relative humidity, electrode and insulting layer physio-chemical characteristics, introduction of catalysts, synergetic technologies, etc., can impact the production and concentration of the reactive and non-radicalized species, ions, electrons, and ultraviolet photons.

However, the potential advantages of generating stable cold plasma are enormous, because the free electrons and free ions created thereby can be selectively transmitted to impact and break down molecules. In other words, it may be beneficial to use cold plasma in a controlled manner so as to generate, disperse, eject, control, or otherwise use free radicals to selectively impact and break down certain molecules. It may also be beneficial to use free radicals to affect or break down molecules into certain constituent elements, and then isolate or otherwise use some of the constituent elements. Thus, cold plasma can be used in various applications, including but not limited to air sterilization, odor neutralization, ozone generation, gas reforming including but not limited to methane and methanol, carbon capture, broad area low-level activation processes, substance creation, plasma-activated water, etc.

As one example, breaking down molecules of a microorganism (single-cell organism) will effectively terminate the organism, thereby operating to sterilize the area in which the microorganism existed, i.e., virus inactivation. In other words, this process can effectively inactivate viruses suspended in the air, in fluids, or on surfaces.

It may be especially beneficial to generate stable cold plasma with high concentrations of Reactive Oxygen Species (“ROS”) and Reactive Nitrogen Species (“RNS”) that are highly effective at inactivating numerous pathogens in the air, on surfaces, and in water. Additionally, reactive species, ions, and electrons are relevant, important, or crucial to decomposing dangerous compounds into non-reactive compounds, chemically inert substances, and basic elements.

As another specific example, it may be beneficial to provide a water treatment device using cold plasma, wherein two electrodes in a double helix shape are disposed around the surface of a dielectric tube, so that a plasma is formed inside the tube between the electrodes. A flow of air through the tube can carry the generated free radicals and other reactive species (ROS and RNS) and direct them into a flow of water, where they can be suspended or held for current or future use in obviating harmful microorganisms or other harmful substances.

It may also be beneficial to provide a water treatment device using cold plasma, wherein two or more electrodes are arranged in specific geometries that are categorized as DBD. For example, one geometry may include two or more planar electrodes separated by one or more dielectric barriers and a spatial gap. Another example may be that of a pair of nested cylindrical electrodes separated by one or more dielectric barriers and a spatial gap. A flow of air or other gas through the gap can carry the generated free radicals and other reactive species (ROS and RNS) and direct them into a flow of water, where they can be suspended or held for current or future use in obviating harmful microorganisms or other harmful substances.

It may be beneficial to integrate the water treatment device with quality control and surveillance technology, which allows for active testing of water quality to further control the water treatment device operation. Some embodiments may be configured to measure gas concentrations within the plasma generating apparatus or dissolved in the treated water, such as ozone, carbon dioxide, carbon monoxide, particulate matter <2.5 microns (PM 2.5), PM 1.0, Formaldehyde, Volatile Organic Compounds (VOCs), Nitrogen Oxides (NOX)2, etc.

### BRIEF DESCRIPTION OF FIGURES

Each figure describes basic features of various methods and apparatuses for generating and/or dispersing free radicals and for separating ionized molecules. Various exemplary aspects of the systems and methods will be described in detail, with reference to the following figures, wherein:

FIG. 1 is a schematic depicting basic features of a plasma generator 10 according to an exemplary embodiment.

FIG. 2 is a perspective view of the plasma generator 10 of FIG. 1.

FIG. 3 is a partial cross-sectional view of the plasma generator 10 of FIG. 2.

FIG. 4 is a top plan view of the plasma generator 10 of FIG. 2.

FIG. 5 is a schematic of an exemplary control circuit for the plasma generator 10 of FIG. 2.

FIG. 6 is a table of different parameters for a plasma generator embodiment with dielectric layers formed of borosilicate glass.

FIG. 7 is a table of different parameters for a plasma generator embodiment with dielectric layers formed of ceramic.

FIG. 8 is a schematic of an exemplary of a liquid processing apparatus 1.

FIG. 9 is a schematic showing some of the basic parameters of the plasma generator 110 of FIG. 8.

FIG. 10 is a top plan view of the plasma generator 110 of FIG. 8.

FIG. 11 is a table of different parameters of tested versions of the plasma generator 110.

FIG. 12 is a schematic of an exemplary embodiment of a liquid processing apparatus 1.

FIG. 13 is an exploded perspective view of an exemplary embodiment of a plasma generator 210.

FIG. 14 is a top plan view of the plasma generator 210 of FIG. 13.

FIG. 15 is a top plan view of an exemplary embodiment of a plasma generator 210 that is a modification of the plasma generator 210 of FIG. 13.

FIG. 16 is a perspective view of an exemplary embodiment of a high-volume liquid processing apparatus 2.

FIG. 17 is a perspective view of an exemplary embodiment of a liquid processing apparatus 3.

FIG. 18 is an exploded perspective view of an exemplary embodiment of the plasma generator 210 according to the liquid processing apparatus 3 of FIG. 17.

FIG. 19 is a top plan view of an exemplary embodiment of the plasma generator 210 of FIG. 18.

### DETAILED DESCRIPTION

The Detailed Description is organized based on the following headings.
Definitions
Plasma Generator
Applications
Overview of the Plasma Generator
Variation of Embodiments
Detailed Explanation
Decreased Ozone Production
Plasma Activated Water
Applications
Overview of the Plasma Activated Water
Variation of Embodiments

### Definitions

It will be understood that, when an element is referred to as being “connected”, or “coupled” to another element, it can be directly connected or coupled to the other element or intervening elements that may be present. In contrast, when an element is referred to as being “directly connected” or “directly coupled” to another element, there are no intervening elements present. As used herein “and/or” includes any and all combinations of one or more of the associated listing items. Further, it will be understood that when an element is “presented” to an entity, it can be presented electronically to the entity through multiple intermediaries or elements of the system. In addition, it will also be understood that when an element is referred to as being “directly presented” to an entity, it is presented electronically through only one intermediary or element of the system. In addition, it will be understood that when an element is presented or directly presented to an entity the presentation may take place on an electronic screen separate from any or all previous electronic screens.

It will also be understood that, although the terms “first”, “second”, etc. may be used herein to describe various elements or components, these elements or components should not be limited by these terms. These terms are only used to distinguish one element or component from another element or component. Thus, a first element or component discussed below may be termed a second element or component without departing from the teachings of exemplary embodiments. Further, it will be understood that the use of “then”, when used to describe connecting two steps of a logical process, indicates that the steps may occur sequentially, but does not preclude the addition of intermediary steps or elimination of one of the steps without departing from the teachings of exemplary embodiments.

Exemplary embodiments are described herein with reference to logical process illustrations that are schematic illustrations of idealized embodiments (and intermediate structures) of exemplary embodiments. As such, variations from the sequence of the illustrations as a result, for exemplary, inclusion of intermediary steps, are to be expected. Thus, exemplary embodiments should not be construed as limited to the particular sequence of logical steps illustrated herein but are to include deviations in the sequence of the steps, for exemplary, from communication of electronic information to remote databases. Thus, the logical steps illustrated in the figures are schematic in nature and their sequence is not intended to limit to scope of exemplary embodiments.

The terminology used herein is for the purpose of describing particular embodiments only and is not intended to be limiting of exemplary embodiments. As used herein, the singular forms “a”, “an”, and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which exemplary embodiments belong. It will be further understood that all terms, such as those defined in commonly used dictionaries, should be interpreted as having a meaning that is consistent with their meaning in the context of the relevant art and will not be interpreted in an idealized or overly formal sense unless expressly so defined herein. As used herein, expressions such as “at least one of”, when preceding a list of elements, modify the entire list of elements and do not modify the individual elements of the list.

Various electrical or electronic elements of the disclosed embodiments, including but not limited to the power supply and control circuitry, are intended to include or otherwise cover all processors, software, or computer programs capable of performing the various disclosed determinations, calculations, etc., for the disclosed purposes. For example, exemplary embodiments are intended to cover all software or computer programs capable of enabling processors to implement the disclosed processes. In other words, exemplary embodiments are intended to cover all systems and processes that configure a document operating system to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed above.

In accordance with the exemplary embodiments, disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl or other sufficient programming languages.

Some of the disclosed embodiments include or otherwise involve data transfer over a network, such as communicating various inputs over the network. The network may include, for example, one or more of the Internet, Wide Area Networks (WANs), Local Area Networks (LANs), analog or digital wired and wireless telephone networks (e.g., a PSTN, Integrated Services Digital Network (ISDN), a cellular network, and Digital Subscriber Line (xDSL)), radio, television, cable, satellite, and/or any other delivery or tunneling mechanism for carrying data. The network may include multiple networks or subnetworks, each of which may include, for example, a wired or wireless data pathway. The network may include a circuit-switched voice network, a packet-switched data network, or any other network able to carry electronic communications. For example, the network may include networks based on the Internet protocol (IP) or asynchronous transfer mode (ATM), and may support voice using, for example, VoIP, Voice-over-ATM, or other comparable protocols used for voice data communications. In one implementation, the network includes a cellular telephone network configured to enable the exchange of text or SMS messages. Some of these and other embodiments utilize a Bluetooth network.

Examples of a network include, but are not limited to, a personal area network (PAN), a storage area network (SAN), a home area network (HAN), a campus area network (CAN), a local area network (LAN), a wide area network (WAN), a metropolitan area network (MAN), a virtual private network (VPN), an enterprise private network (EPN), Internet, a global area network (GAN), and so forth.

Central database systems may include a network server for communicating with the various remote computer systems. Communication to the network may be over the Internet, other networks, telephone, or other suitable means. The central database systems further include a central database and database server for storing and retrieving information. The network server can be operated by software that allows communication with the remote computer systems and transfers information to and from the database server for maintenance of the database and for providing patient-specific information.

Reference will now be made in detail to embodiments, examples of which are illustrated in the accompanying schematics, wherein like reference numerals refer to the like elements throughout. In this regard, the present embodiments may have different forms and should not be construed as being limited to the descriptions set forth herein. Accordingly, the embodiments are merely described below, by referring to the figures, to explain exemplary embodiments of the present description. It is also important to note that various elements and features may be used interchangeably among the different disclosed embodiments, and so the various elements and features may be combined to achieve new embodiments.

Plasma Generator

Embodiments are intended to include or otherwise cover a variety of structures and processes for generating cold plasma for any possible advantageous use, e.g., so that the free electrons and free ions created thereby can be selectively transmitted to impact and break down molecules. Some of the disclosed plasma generator embodiments constitute large-area multi-layer dielectric barrier plasma discharge units or “plasma generators” or “reactors”; however, other embodiments can cover other types of units.

Some of the generated plasmas as disclosed herein are atmospheric pressure, low-temperature plasma (hereinafter “cold plasma” or “non-equilibrium plasma” or “stable plasma” or “plasma”). However, embodiments are intended to include or otherwise cover other types of plasmas that may be beneficial.

Some of the disclosed plasma generators include a first electrode covered (e.g., in part) with a dielectric layer, and a second electrode similarly covered (e.g., in part) with a dielectric layer, wherein these electrodes/dielectrics are separated from each other by a gap in which cold plasma is generated. However, embodiments are intended to cover or otherwise include other structures for generating the cold plasma, such as including but not limited to only one of the electrodes being covered (e.g., in part) with a dielectric layer, or alternatively multiple dielectric layers being disposed between a pair of electrodes. Moreover, some embodiments can include multiple plasma generators “stacked” on top of one another.

In some of these embodiments, stable cold plasma is generated based on the following factors: 1) height of gap separating the electrodes/dielectrics; 2) voltage of AC electrical power supplied to the electrodes; and 3) frequency of electrical power supplied to the electrodes. However, other factors can affect the stable cold plasma generation of some embodiments, including the materials from which the electrodes and dielectric layers are formed, the relative permittivity and strength of the dielectric layer, etc. For example, in order to generate stable cold plasma, the output voltage can be controlled based on certain structural parameters of the plasma generator, such as the vertical distance separating the electrodes, the materials from which the electrodes are formed, the plane size and thickness of the electrodes, etc.

In addition, embodiments are intended to cover or otherwise include any combination of the above factors that achieves the generation of stable cold plasma, including but not limited to the exemplary ranges specifically disclosed herein. Still further, embodiments are intended to include or otherwise cover any methods, processes, or structures for supplying, varying or otherwise modifying the electrical power to the electrodes having appropriate voltages and frequencies for generating the stable cold plasma.

A few exemplary plasma generator embodiments are disclosed below, however as indicated above, embodiments are intended to cover many different variances in structure and function from the specific elements and processes disclosed below.

Applications

Embodiments are intended to cover various structures of plasma generators for creating or otherwise generating free electrons and free ions, such as to selectively transmit the generated free electrons and free ions to impact and break down molecules for any useful purpose. Embodiments are also intended to be configured for use with any advantageous or otherwise beneficial applications of the stable cold plasma and the free electrons and free ions generated thereby. For example, embodiments are intended to include or otherwise cover any advantageous or otherwise beneficial application of using the generated free electrons and free ions to break down or otherwise modify molecules.

For example, embodiments can be used in various contexts related to disinfection, including viral inactivation. This usage may be especially poignant based on the fact that droplet transmission is associated with the spread of coronavirus and similar pathogens including other viruses, pathogenic bacteria, mold, poisons, gases, mycotoxins (aerosol infection) constitute one of the main routes of infection. Thus, it may be beneficial to structure or otherwise use the plasma generator embodiments disclosed herein to contact air that may include such pathogens and thereby sterilize the air to reduce, minimize, or prevent the transmission and spread of the pathogens.

Embodiments are intended to include or otherwise cover any structures or processes that enable or otherwise adapt the disclosed plasma generators for such sterilization usages. For example, the disclosed plasma generators can be adapted to operate in conjunction with equipment configured to supply air to the generated plasma, and then to export the sterilized air back into the environment in which the air was originally obtained.

For some of these embodiments that operate as air sterilizers, additional structures may be provided for enhanced sterilization, such as apparatus for increasing turbulence of air upon entering an inlet into the gap in which the cold plasma is generated. The increased turbulence increases the contact rate of the air with the electrons and ions to enhance sterilization.

A number of electrodes for generating cold plasma can be arranged into a matrix to scale the sterilization process. In other words, a plurality of plasma generating units (each of which can be planar), can be stacked and otherwise provided to ensure that collected ambient air is efficiently mixed with the plasma to enhance sterilization.

However, embodiments are intended to cover or otherwise include any other useful application of creating or otherwise generating free electrons and free ions, including but not limited to selectively transmit the generated free electrons and free ions to impact and break down molecules.

For example, some embodiments are directed to breaking down greenhouse gases in any possible context, such as in the exhaust of industrial and manufacturing facilities (e.g., coal plants, cement manufacturing, etc.), internal combustion engine operations (e.g., automobiles), etc. Still other embodiments are directed to breaking down molecules via a liquid, including water-based liquids, such as in the contexts of waste-water treatment, using water to sterilize various things including but not limited to cloths, farm animals, etc. Other embodiments are directed to still other applications, such as sterilizing currency.

Overview of the Plasma Generator

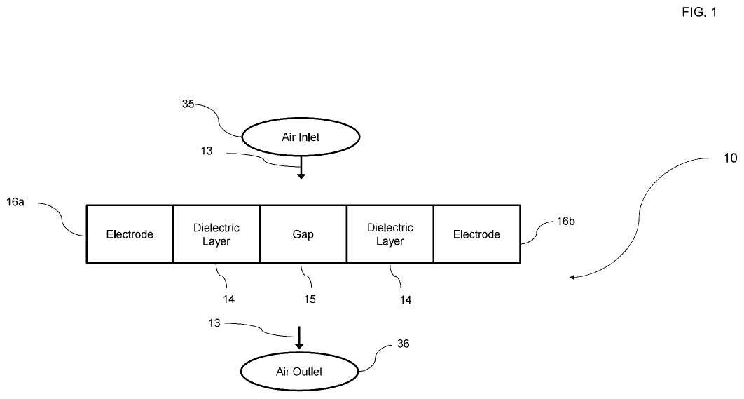

FIG. 1 is a schematic depicting basic features of a plasma generator 10 according to an exemplary embodiment wherein the various elements are schematically shown in the context of their relative positioning. As shown in FIG. 1, the plasma generator 10 includes a pair of electrodes 16a, 16b at opposing sides of the plasma generator 10.

In some of the disclosed embodiments, the electrodes 16a, 16b are each planar (flat) conductive metal plates. However, embodiments are intended to include or otherwise cover any type (including any material, shape, size, etc.) of electrical conductor that is usable to make contact with a metallic part of a circuit configured to generate stable cold plasma. For example, in some embodiments, the electrodes 16a, 16b are solid metal or a punched solid metal while in other embodiments the electrodes 16a, 16b are formed as a mesh that defines gaps therewithin. Also, it is intended that the electrodes 16a, 16b can be formed of any related art, known, or later developed material that operates as a similar electrical conductor suitable for the purpose of generating stable cold plasma. In some embodiments, the electrodes 16a, 16b may be mirror-polished metal, including stainless steel.

In some embodiments, the electrode is the circuit element that allows current to pass through its volume to its surface of the electrode. The electrons stay confined to the surface and cannot exit due to the dielectric material directly opposite the incoming current. Similarly, the electrons are allowed to pass from the surface of the electrode through its volume, without any electrons passing through the dielectric material to the electrode surface opposite an outgoing current. In other words, the electrons can pass freely through the volume of the electrodes, but are unable to pass through (or are otherwise impeded from passing through) the volume of the dielectric material when the applied voltage is less than the dielectric strength of the dielectric material.

In some embodiments, the electrodes 16a, 16b have the same structure, size, and shape. For example, both electrodes can be formed into the same or substantially the same size and planar shape, where each defines a rectangle, square, etc., in top plan view. However, in other embodiments, the electrodes 16a, 16b can have different shapes and sizes, and can be formed of different materials.

As shown in FIG. 1, a pair of dielectric layers 14 is disposed between both electrodes 16a, 16b. However, in some embodiments, only one dielectric layer 14 is disposed between the electrodes 16a, 16b. Also in some embodiments, each dielectric layer 14 is adhered onto one, or both, of the electrodes 16a, 16b. In some embodiments, each dielectric layer 14 is disposed directly on one of the electrodes 16a, 16b, such as in cases where the dielectric layer 14 is adhered or otherwise integrally formed onto the electrode 16a, 16b.

However, in some other embodiments, each dielectric layer 14 can be separated from the nearest electrode 16a, 16b, such as where an empty gap extends therebetween or alternatively where another element extends therebetween. More specifically, an adhesive material may be provided to separate the electrode 16a, 16b from the dielectric layer 14. In other words, embodiments are not limited to structures where all of the dielectric layers are necessarily in direct contact with electrodes, and some embodiments may even cover structures where other elements are disposed between electrodes and dielectric layers.

Embodiments are intended to include or otherwise cover the dielectric layer(s) 14 as constituting any electrical insulator that can be polarized by an applied electric field, such that if the dielectric material is placed in an electric field, then electric charges do not flow through the material as they do in an electrical conductor (in this case the electrodes 16a, 16b), so as to thereby be usable with the other elements of the plasma generator 10 to generate stable cold plasma.

More specifically, the dielectric layer(s) 14 are envisioned to be formed of any related art, known, or later developed material without (or with reduced) loosely bound (or free) electrons that may not drift through the material, and that instead shift (slightly), from their average equilibrium positions, causing dielectric polarization. Because of this dielectric polarization, positive charges are displaced in the direction of the field and negative charges shift in the direction opposite to the field (for example, if the field is moving parallel to the positive x axis, the negative charges will shift in the negative x direction, so as to create an internal electric field that reduces the overall field within the dielectric itself. In embodiments where the dielectric is composed of weakly bonded molecules, those molecules not only become polarized, but also reorient so that their symmetry axes align to the field. Common dielectric materials include glass, ceramic, and most plastics.

In some embodiments, the dielectric layers 14 have the same structure, size, and shape. For example, both dielectric layers 14 can be formed into the same or substantially the same size and planar shape, where each defines a rectangle or square in top plan view. However, in other embodiments, the dielectric layers 14 can have different shapes and sizes and can be formed of different materials.

In some embodiments, there can be one or two dielectric layers between each pair of electrodes, yielding beneficial results when there are two dielectric layers between each pair of electrodes. The number of electrodes can also vary, wherein the horizontally aligned electrodes are adhered to and stacked vertically with dielectric layers and fluid gaps between every stacked electrode such that first and second electrodes serve as top and bottom electrodes to adjoin one plasma gap (hereinafter “plasma gap” or “gap”) in a stack of electrodes and dielectric layers to create multiple plasma gaps.

Embodiments are intended to include any rotational orientation with respect to Earth's gravity and any mention of the terms “vertically” or “horizontally” are meant to be used in a relative frame of reference rather than an absolute frame of reference. For example, “vertical stacking” describes the positioning of flat planar electrodes in a stacking direction that is perpendicular to the planes of the electrodes. However, the planes of the electrodes can make any angle with the direction of Earth's gravity, so a “vertically stacked” set of electrodes could be considered to be either vertically stacked, horizontally stacked, or stacked in a slanted direction with respect to Earth's gravity.

Embodiments are intended to include or otherwise cover any number of electrodes. In some embodiments, the number of electrodes can be configured to be between two and fifty-one, where the number of gaps between electrodes can be between one and fifty, yielding beneficial results with the number of fluid gaps between five and twenty, and still further at ten. However, embodiments are intended to include or otherwise cover any number of electrodes and/or gaps. However, as indicated above, some embodiments can include more than fifty-one electrodes.

As shown in FIG. 1, a gap 15 is defined between the dielectric layers 14. In some embodiments, the range of the gap 15 between the dielectric layers is approximately 1.5 mm to approximately 3.0 mm. In some of these embodiments, the gap 15 between the dielectric layers is approximately 1.75 mm to approximately 2.25 mm. In some of these embodiments, the gap 15 between the dielectric layers is approximately 2.0. However, embodiments are intended to include or otherwise cover gaps of any width or height applicable for any conceivable application, material, etc.

Electrical power at a predetermined AC voltage and frequency can be applied to the first and second electrodes 16a, 16b, such that, based also on the height of the gap 15 and other factors, stable cold plasma is generated within the gap 15. Other factors that affect the generation of cold plasma within the gap 15 include (but are not necessarily limited to) material from which the electrodes 16a, 16b are formed, plane size and thickness of the electrodes 16a, 16b, aspects of the dielectric layer 14, etc. Thus, in some embodiments, in order to generate stable cold plasma, the output voltage needs to be controlled based on certain structural parameters of the plasma generator 10, such as distance between electrodes 16a, 16b (i.e., gap 15), the material from which the electrodes 16a, 16b are formed, the plane size and thickness of the electrodes 16a, 16b, aspects of the dielectric layer 14, etc.

In other words, stable cold plasma can be generated within the gap 15 based on at least the following factors: 1) height of gap 15 separating the electrodes/dielectrics; 2) voltage of AC electrical power supplied to the electrodes 16a, 16b; 3) frequency of electrical power supplied to the electrodes 16a, 16b; 4) structural characteristics of the plasma generator 10 including but not limited to the above structural factors; 5) dielectric constant (permittivity) of the dielectric material; 6) thickness of the dielectric material; and/or 7) composition of the fluid in the plasma gap. Some embodiments use or otherwise incorporate semiconductors (in lieu of transformers), e.g., silicon carbide high voltage chip, such as a chip capable of generating 3 kV or more of applied plasma voltage).

FIG. 1 also shows an airflow path 13 that enables air to flow from an air inlet 35, into the gap 15 and thus in contact with the free electrons and free ions generated by the cold plasma, and then out of an air outlet 36. Molecules that come into contact with the free electrons and free ions of the cold plasma are thereby broken down or otherwise altered.

Multiple plasma generators 10 that have the same or similar structures to those discussed above can be provided to scale the amount of cold plasma generated. In some of these embodiments, the layers of electrodes 16a, 16b and dielectrics 14 can be stacked so as to result in a vertically aligned structure, wherein a separate gap 15 separates each layer of an electrode 16a, 16b laminated with a dielectric layer 14.

Some of the embodiments create stable cold plasma by focusing on various parameters, calculations, and the application. For example, some embodiments are directed to a multi-layered dielectric barrier plasma discharge generator with a gap of approximately 2 mm. Some of these embodiments are equipped to sterilize and decompose large volumes of atmospheric, indoor air and greenhouse gas emissions. The materials and parameters of elements of the generator of these embodiments may change depending on the composition of the fluid (i.e., gas) entering the plasma field.

Some embodiments of the plasma generator 10 for indoor air sterilization require less voltage than other embodiments that are applied for greenhouse Fluid Decomposition, such as an industrial gas decomposition apparatus. In general, the composition of atmospheric/indoor air is 78% Nitrogen, 21% Oxygen, 0.93% Argon, and 0.04% Carbon Dioxide—the remainder being trace amounts. To create sufficient stable plasma to sterilize indoor air, some embodiments of the plasma generator 10 produce a minimum voltage, ranging from 3 kV-7.5 kV, based on bond energies and electron Volts required to separate Hydrogen-Oxygen bonds (1.23 eV), reforming into hydroxyl radicals (OH), hydrogen peroxide (H2O2), as well as sufficient energy to eradicate specific pathogens (i.e., viruses, bacteria, fungi, etc.). In the context of carbon decomposition, the voltage may be increased to allow the electrons to separate carbon oxygen bonds (5.45 eV) in carbon dioxide molecules.

In some of these embodiments, once the voltage range is established, the capacitance requirements of the plasma generator can be calculated. Some embodiments for indoor air sterilization are designed with a 2 mm plasma gap, 2 mm dielectric layer 14 with a minimum relative permittivity of 4. Some of these embodiments use borosilicate glass as the insulating layer, which includes a relative permittivity of 4.7 at 60 Hz and minimum dielectric strength of 30 kV/mm (to ensure there is no or reduced dielectric breakdown).

In some embodiments, the borosilicate glass can be treated to resist higher temperatures. For example, the borosilicate glass can be treated to become tempex glass that can resist temperatures up to 600 degrees Celsius.

In some of these embodiments, once the voltage range is established, the capacitance requirements of the plasma generator can be calculated. Some embodiments for indoor air sterilization are designed with a 2 mm plasma gap, 2 mm dielectric layer 14 with a minimum relative permittivity of 25. Some of these embodiments use ceramic as the insulating layer, which includes a relative permittivity of 25 at 60 Hz and minimum dielectric strength of 24 kV/mm (to ensure there is no or reduced dielectric breakdown).

Some embodiments use a 6 cm×9 cm electrode 16a, 16b formed of aluminum, with a total area of 54 cm2 per electrode. Other embodiments use a 30 cm×30 cm electrode 16a, 16b formed of aluminum or other conductive material thereby providing a total area of 900 cm2 per electrode. Total separation distance between electrodes 16a, 16b for some of these embodiments is 6 mm. Once standards are established, the electrical components necessary to convert inputted DC 12 V to the required AC 3 kV-15 kV can be determined. Based on the air flow requirements calculated using streamer discharge, length and width of electrode 16a, 16b and the plasma Gap 15, additional Plasma Gaps 15 can be added while maintaining the same voltage requirements. At such time that stable plasma is no longer generated due to the cumulative area (cm2) of the electrode 16a, 16b exceed the applied voltage, revisions can be made to the voltage, dielectric layers, and plasma Gap 15 parameters.

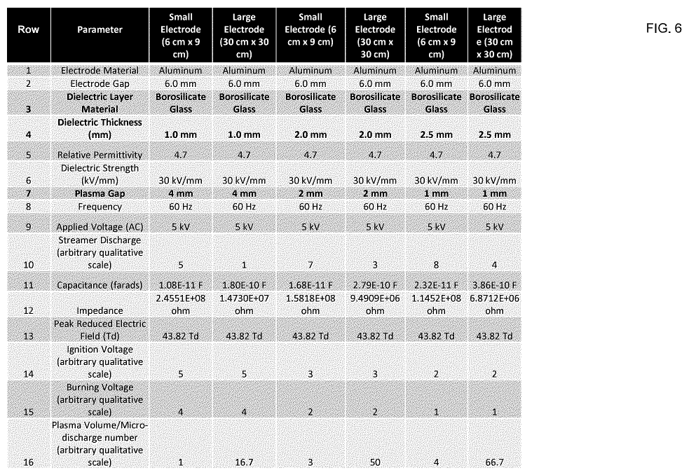

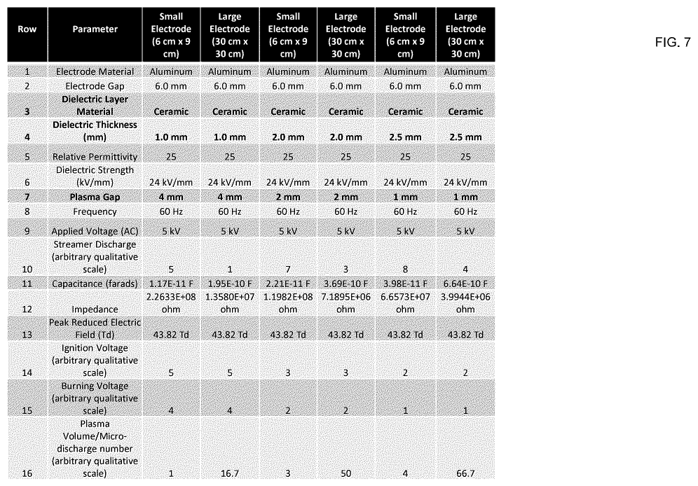

All of the embodiments are intended to cover applications where any fluid is disposed in the Gap 15. For example, the fluid in the gap can be “atmospheric air” (such as in the context of the tables of FIG. 6 and FIG. 7, any other gas or gaseous material (including but not limited to products and byproducts of industrial or commercial processes, etc.), any fluid in liquid form, any slurry, etc. In fact, the fluid is not even limited to the before-mentioned fluids and is intended to cover any substance disposed in the Gap 15.

### Variations of Embodiments

Embodiments are intended to include or otherwise cover any methods and apparatus for creating stable cold plasma. Some of these embodiments are directed to generating stable cold plasma under atmospheric conditions where ambient fluid and small biological particles enter and interact with the plasma. In some of these embodiments, some or all of the small biological particles are sterilized by interaction with the stable cold plasma. However, other embodiments generate stable cold plasma for interaction with other fluids, such as fluids that include various gas species and concentrations.

Some embodiments include or otherwise cover relatively small electrode plates (such as, for example, electrode plates that are defined by a width of 6 cm and length of 9 cm), where a first electrode may have a dielectric layer that is formed of insulating material, such as glass or other dielectric materials, on its bottom surface. A second electrode plate of approximately the same size as the first electrode plate (in some embodiments 6 cm×9 cm) may also have a dielectric layer on its top side formed of the same dielectric material as the dielectric layer of the first electrode. The stacking configurations of some of these embodiments may define a constant 6 mm separation between the first and second electrode plates.

In some of these embodiments, this 6 mm separation includes the two dielectric layers and a gap in which stable cold plasma is generated. In some of these embodiments that include the 6 mm separation between electrodes, the dielectric layer may be 2 mm thick on each electrode plate, resulting in a 2 mm gap in which plasma is generated. In some of these embodiments, the stable cold plasma is generated upon AC 5 kV being applied to the electrodes at 60 Hz.

Some other embodiments include or otherwise cover relatively large electrode plates (such as, for example, electrode plates that are defined by a width of 30 cm and length of 30 cm) in the same or similar configurations as described above regarding the relatively smaller (e.g., 6 cm×9 cm) electrode plates. Some of these embodiments that include relatively larger electrodes may be especially beneficial because they may generate a relatively larger volume of stable cold plasma and enable a relatively larger volume of fluid to flow through the gap.

More specifically, embodiments that also include a constant 6 mm separation between the first and second electrodes, where the thickness of each dielectric layer is 2 mm and the plasma gap is 2 mm, enable a relatively increased volume of plasma generated in the gap between the dielectric layers as well as a relatively increased volume of fluid flowing through the plasma gap. In these embodiments, relatively larger electrode plates may be beneficial because they increase the capacitance of the system, yet the applied voltage and frequency will remain the same as that used for relatively small plate configurations.

In some of these and other embodiments, the dielectric layer thickness may be less than 2 mm. For example, if the dielectric layers of these embodiments has a thickness of 1 mm or 1.5 mm, then the plasma gap will increase, assuming a fixed separation between electrode. In other words, the distance between the electrodes will remain the same but the gap size may differ if the dielectric material thickness changes, creating a bigger or smaller gap where plasma is generated. In some of these embodiments, this increased gap may cause a slight increase in capacitance, and the burning voltage may also increase due to a smaller dependence on the surface electron emission properties of the dielectric layer on both the first and second electrode plates. In some of these embodiments, these combined effects may cause the total amount of plasma generated through an AC cycle, as well as the volume of the plasma at any one time, to both decrease.

In some embodiments, if the plasma gap decreases due to a relatively thicker dielectric layer within the 6 mm separation between the first and second electrode plates (or for some other structural reason), then the capacitance and the burning voltage may decrease. In some of these embodiments, these effects may combine to allow for the total amount of plasma generated through an AC cycle as well as the volume of the plasma at any one time to both increase. These qualitative changes may be constant between different embodiments, such as the relatively small electrode plate configurations (e.g., 6 cm×9 cm) and the relatively large electrode plate configurations (e.g., 30 cm×30 cm). However, it is important to note that embodiments are not limited to these electrode plate sizes, and embodiments are intended to include or otherwise electrode plates of any size and shape that is usable to generate or help generate stable cold plasma.

In some embodiments, changes in relative permittivity, dielectric strength, and/or surface charge properties occurs if the insulating material of the dielectric layer changes, for example from glass to ceramic. This change may alter the capacitance of the system, the production rates of each chemical species in the plasma, and in some embodiments force a change to the applied voltage. However, in some of these embodiments, the frequency and gap size between each electrode plate, such as at an electrode separation of 6 mm, will remain constant. These changes are constant between relatively small electrode plate configurations (e.g., 6 cm×9 cm) and relatively large electrode plate configurations (e.g., 30 cm×30 cm).

In some embodiments, small electrode plates (e.g., 6 cm×9 cm) may be configured within larger (e.g., 12 cm×15 cm) plates that are made of the same material as the dielectric layer material. In some embodiments, large electrode plates (e.g., 30 cm×30 cm) may be configured within larger plates (e.g., 50 cm×50 cm) that are made of the same material as the dielectric layer material. Embodiments with these configurations include larger creepage areas as per Japanese, US, European, or other standards.

### D. Detailed Explanation

The plasma generator 10 of FIG. 1 is disclosed above at a high level and will be discussed in much more detail below.

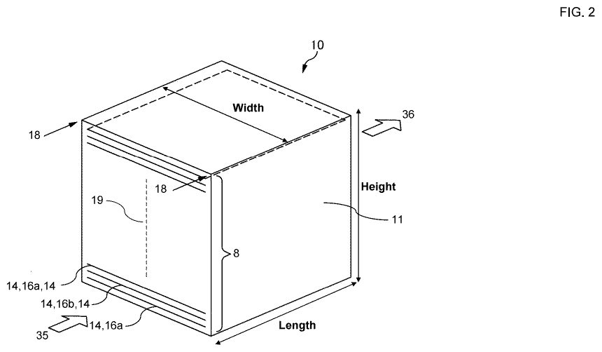

FIG. 2 is a perspective view of the plasma generator 10 of FIG. 1. FIG. 2 specially shows an exterior housing 11 of the plasma generator 10 that houses multiple stacked electrodes 16a, 16b, which are each laminated with a dielectric layer 14, and then vertically separated by gaps 15 from other electrodes 16a, 16b laminated with dielectric layers 14. These stacked layers extend vertically to a height 8 of the housing 11. Thus, the plasma generator 10 of FIG. 2 is formed in a rectangular parallelepiped shape having a rectangular plane as a whole.

As specifically shown in FIG. 2, each horizontal line represents an electrode 16a, 16b, which is laminated with either one or two dielectric layers 14. For example, the bottom horizontal line constitutes an electrode 16a laminated on its top surface with a dielectric layer 14, but not its bottom surface because it constitutes the lowermost layer. Similarly, the top horizontal line constitutes an electrode 16a laminated on its bottom surface with a dielectric layer 14, but not its top surface because it constitutes the topmost layer. However, the horizontal lines representing the layers in the middle, including the second lowermost layer, include an electrode 16b with both the top and bottom surfaces that are each laminated with a dielectric layer 14. Dashed line 19 in FIG. 2 represents multiple such layers extending vertically along the height 8 of the housing 11 in a vertically disposed central area of the housing 11. Each of the dielectric layers 14 are separated by a gap 15 such that many such gaps 15 are defined within the plasma generator 10.

An air inlet 35 enables air to enter a front end of the multiple stacked gaps 15, wherein plasma is generated if electrical power is applied to the electrodes 16a, 16b. Air is taken in from the front side of the plasma generator 10, passes through a plurality of flow paths defined by the gaps 15, and is exhausted from the rear of the plasma generator 10. In other words, each of the gaps 15 constitute air passages formed in the plasma generator 10, which in the embodiment of FIG. 2 is defined as a flat slit-shaped open area.

Stable cold plasma is generated within those gaps 15 so that the air contacts or otherwise communicates with the free ions and free electrons generated by the cold plasma, thereby causing molecules in the air to break down or otherwise be altered. Air with the broken down or altered molecules then exits the plasma generator 10 at the air outlet 36.

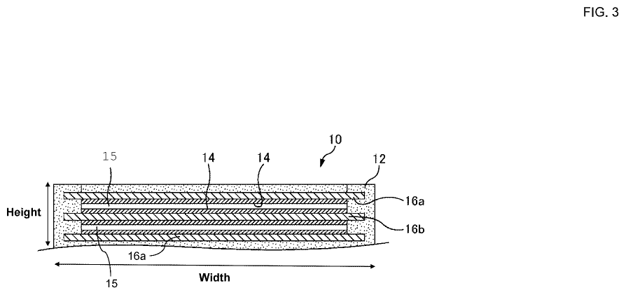

FIG. 3 is a partial cross-sectional view of the plasma generator 10 of FIG. 2, which shows three such electrode/dielectric layers with two separate gaps 15 defined therebetween. As shown in FIG. 3, the top layer includes an electrode 16a that is laminated with a dielectric layer 14 on its bottom surface, which is then separated by a gap 15 from the adjacent layer disposed immediately there below, which itself includes an electrode 16b that is laminated by dielectric layers 14 on both its top and bottom surfaces.

The FIG. 3 embodiment shows the middle electrode 16b separated from each of its neighboring electrodes 16a (above and below) by two separate dielectric layers 14. In particular, a dielectric layer 14 is provided on both of its top and bottom surfaces, and a separate dielectric layer 14 on each of the neighboring electrodes 16a. However, embodiments are intended to cover structures where neighboring electrodes 16a, 16b are only separated by a single dielectric layer 14.

The electrodes 16a, 16b (with their attached dielectric layers 14) are held in place by a spacer 12 disposed at opposite width-wise ends. The spacer 12 holds the electrodes 16a, 16b in place so as to define the gaps 15, each extending vertically to a predetermined distance, between the dielectric layers 14. As shown in FIG. 3, the spacer 12 extends above, below, and to the side ends of each of the electrodes 16a, 16b, wherein the side ends are cantilevered beyond the dielectric layers 14 in the width-wise direction. The spacer 12 can also be disposed above the top and below the bottom of the stacks of electrodes/dielectric layers to further hold the structure together.

The spacer 12 can be formed of a single contiguous or unitary element, or alternatively can be formed by multiple elements. The spacer 12 is formed of an insulating material, similar or including (but not limited to) the materials from which the dielectric layer 14 are formed, and is intended to include any related art, currently known, or later developed material(s). The spacer 12 can be adhered to the dielectric layer 14 for the electrodes 16a, 16b.

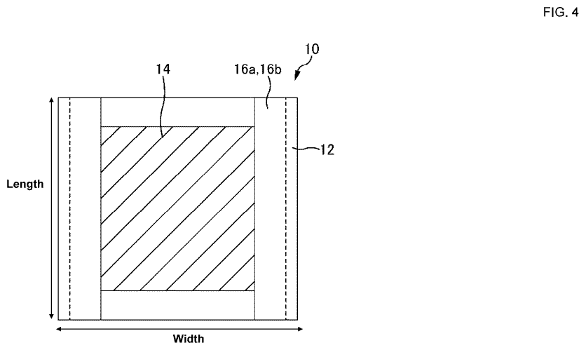

FIG. 4 is a top plan view of the plasma generator 10 of FIG. 2. As shown in FIG. 4, the spacer 12 is disposed at certain locations around the perimeter of the electrodes 16a, 16b, thereby holding them in place while also constituting an insulator. FIG. 4 specifically shows the spacer 12 extending around the sides of the electrodes 16a, 16b. Also as shown in FIG. 4, the dielectric layers 14 do extend as far as the electrodes 16a, 16b in the widthwise direction, such that the face of each of the side ends of the dielectric layers 14 abuts the spacer 12.

In the embodiments of FIGS. 1-4, each of the electrodes 16a, 16b can be a metal or mesh plate in the shape of a thin sheet that is rectangular or square in top and bottom plan views, wherein the top and bottom surfaces are flat or planar. However, the electrodes 16a, 16b can be formed into any shape that may be beneficial for the application at issue. Thus, the width and length of each electrode 16a, 16b is each longer than its height. In some of these embodiments, the electrodes 16a, 16b are formed of an aluminum plate, but may be formed of any other suitable conductive related art, known, or later developed material, including but not limited to stainless-steel plate, silver, gold, magnesium alloy, etc.

Embodiments are intended to cover electrodes 16a, 16b of any size depending on application. It is beneficial to avoid bending of the electrodes 16a, 16b, such as that may result from widthwise interior section extend into the adjacent gap 15, which may occur based on mass and gravity and become particularly acute as the length of the electrode 16a, 16b increases. The size (including length) of the electrodes 16a, 16b can be increased by using stronger materials that resist such bending.

In the embodiments of FIGS. 1-4, each electrode is laminated, at least in part, with a dielectric layer 14 that is formed of a solid insulating resin, such as borosilicate glass, fiberglass, aluminum oxide, ceramics, Pantex glass, polyimide film, etc., allowing for high breakdown voltage. A creepage distance is located around each electrode 16a, 16b, allowing for high voltage to run through it. The size of the creepage distance as shown on FIG. 4 is based on industrial standards, such as those in the United States, Europe, or Japan.

In the embodiments of FIGS. 1-4, the dielectric layers 14 (insulating layers), at least in part, can be formed as a layer of glass, such as from the materials disclosed above or other materials. The dielectric insulating layer 14 operates to stabilize the plasma generated within the gap 15, and can include various other materials, such as quartz, ceramics, enamel, or similar materials. The thickness of each dielectric layer 14 can affect the stable cold plasma generation and so can fluctuate. For some embodiments, the thickness of each dielectric layer 14 produces a 2 mm gap 15 between adjacent dielectric layers 14.

In the embodiments of FIGS. 1-4, the dielectric layers 14 are provided on the surfaces of opposing electrodes 16a, 16b, and the dielectric layers 14 are supported so as to be parallel to each other while being separated by spacers 12. The size of the spacers 12 (which operate as spacing members) is determined by the thickness of electrodes 16a and 16b, dielectric layers 14, and gap 15. The spacers 12 can be formed from any electrically insulating resin material, such as glass (similar to that used for the dielectric layers 14 as described above).

As discussed above, stable cold plasma can be generated within the gap 15 based on the following factors: 1) height of gap 15 separating the electrodes/dielectrics; 2) voltage of AC electrical power supplied to the electrodes 16a, 16b; 3) frequency of electrical power supplied to the electrodes 16a, 16b; and/or 4) structural characteristics of the plasma generator 10. Some of these structural factors that impact creating stable cold plasma include aspects of the dielectric layers 14, such as break voltage, i.e., the dielectric strength or kV per mm, and the relative permittivity. In some embodiments, creating stable cold plasma is enhanced by providing for a higher relative permittivity.

In the embodiments of FIGS. 1-4, the height of each gap 15 is set to be approximately 2 mm, and the thicknesses or height of each of the electrodes 16a, 16b is set to be approximately 2 mm. As schematically shown in FIG. 2, an appropriate number of gaps 15 (for example, about 30^{˜}40 layers) are provided in the height direction (height 8) of the plasma generator 10. However, any number of layers can be provided depending on the capacity required.

In some of the embodiments of the structures shown in FIGS. 1-4, the width of each gap 15 can be 100 mm-400 mm (10 cm-40 cm). However, it is especially beneficial for a width of the gap 15 to be 250 mm-350 mm (25 cm-35 cm), and still better if the width of the gap 15 is approximately 300 mm (30 cm). However, in other embodiments, the width of the gap 15 can be reduced to 30 mm-90 mm (3 cm-9 cm). In those embodiments, it may be especially beneficial for the width of the gap 15 to be 45 mm-75 mm (4.5 cm-7.5 cm), and still better if the width of the gap 15 is approximately 60 mm (6 cm). The depth of each gap 15 along the flow direction is approximately 300 mm (30 cm). However, the size of each gap 15 can be modified as per application use, such as for the purpose of increasing the volume of fluid that can be processed while maintaining electricity levels that result in stable plasma generation.

As shown in FIG. 3, the spacers 12 (formed of an electrically insulating resin material, similar to the glass used for the dielectric layer) can be formed to a height that exceeds the combined heights of the electrodes 16a, 16b, dielectric layers 14, and the gap 15. In other words, in some embodiments, the spacers 12 extend adjacent side ends (in the widthwise direction) of the electrodes 16a, 16b and dielectric layers 14, and also above (in the height direction) cantilevered side ends of the electrodes 16a, 16b, which provides structural stability of the generator 10, especially in embodiments that include numerous stacked layers. For example, in an embodiment that includes two electrodes 16a, 16b that are each 2 mm in height, two dielectric layers 14 that are each 2 mm in height, and a gap that extends between the dielectric layers 14 to a height of 2 mm, it may be beneficial for the spacer 12 to extend to a height exceeding 7 mm. Some embodiments may include materials, including but not limited to materials used for the purpose of adhesion of components, placed between the electrodes 16a, 16b the dielectric layers 14, and the spacer 12, which may increase the gaps between these components. In this case, it may be beneficial for the spacer 12 to extend to a height exceeding 8 mm or even 9 mm.

The size and number of dielectric layers 14 and electrodes 16a, 16b has a direct correlation to the voltage used. Thus, some embodiments increase the spacer 12 size to handle higher voltages i.e., to allow for proper creepage distance.

In the embodiments of FIGS. 1-4, stacking electrodes/dielectric layers helps create stable plasma generated in the gaps 15 defined therebetween. Further, the stacked design allows for more gaps 15 and thereby enables more fluid to pass through the plasma generator 10, which is beneficial for many applications. Embodiments are intended to include or otherwise cover any number of stacked electrode/dielectric layers separated from each other by gaps 15, including but not limited to 100 layers, 11 layers, etc. For example, some embodiments include stacked units with 11 electrodes 16a, 16b, resulting in 10 gaps 15 in which plasma is generated. It may be beneficial to provide enough electrodes 16a, 16b to define 20 gaps 15 in which stable cold plasma is generated. However, as indicated above, embodiments can include any number of stacked electrode/dielectric layers separated from each other by gaps 15.

In the embodiments of FIGS. 1-4, efficiencies of plasma generation are the same or substantially the same regardless of how much fluid is processed through the gaps 15 of the plasma generator 10. However, effectiveness of the plasma generation does change and is affected based on the type of fluid passing through the plasma generator 10. In other words, the type of fluid passing through the plasma generator can lead to changing any of the following parameters: dielectric thickness, dielectric material, electrode material, electrode gap, plasma gap, applied voltage, frequency of applied voltage, etc.

In the embodiments of FIGS. 1-4, applying a predetermined AC voltage at a predetermined frequency between the electrodes 16a, 16b generates cold plasma in the gaps 15 between the dielectric layers 14, which is based on the dielectric barrier discharge induced in the plasma gap 15. This acts to ionize CO2, for example, contained in air, disposed in the gap 15 and in contact with the plasma, to separate the carbon atoms and oxygen atoms. Specifically, a CO2 molecule receiving energy from the plasma ionizes into a positively charged carbon atom (hereinafter “C+”), which has lost electrons in the outermost shell, and a negatively charged oxygen atom (hereinafter “O—”), which has received electrons from the carbon atom. Ultimately, the ionized C+ and O— favor being recombined and returning to stable CO2, and so a separation unit 21 described in the following section is provided adjacent to the downstream side of the plasma generator 10 or is combined with the plasma generator 10 to isolate the ionized C+ and O— before they naturally recombine.

Cold plasma acts on air and water vapor (i.e., fluid) to generate reactive oxygen species including various radicals, such as singlet oxygen (1 O2), ozone (O3), hydroxyl radical (OH), superoxide anion radical (O2-), hydroperoxyl radical (HO2) and hydrogen peroxide (H2O2). The fluid passing through each gap 15 of the plasma generator 10 flows while being in contact with the generated plasma by continuously spreading in each planar shaped gap 15. Microorganisms, such as viruses and bacteria, contained in the surrounding air sucked into each gap 15 are very quickly destroyed, such as within microseconds, by contacting the cold plasma. A mixture of a multi-plasma gas containing the reactive oxygen species inactivates the viruses and sterilizes the microorganisms. Thus, the plasma generator operates to effectively sterilize the ambient air entering the gaps 15.

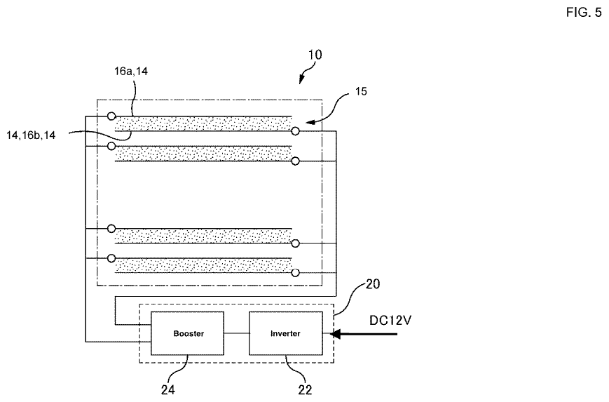

FIG. 5 is a schematic of an exemplary control circuit for the plasma generator 10 of FIG. 2. As shown in FIG. 5, a power supply 20 supplies AC electrical power at a predetermined voltage and frequency to the electrodes to generate cold plasma within the gaps 15. The power supply includes and is intended to cover any related art, known, or later developed equipment for performing the above operation.

The power supply includes an inverter 22 and a booster 24, which is connected to each electrode 16a, 16b. DC 12 V is input to the inverter 22 of the plasma power supply unit 20 from an external power source. The inverter 22 outputs an AC voltage controlled in accordance with the input DC voltage and supplies it to the booster 24. The inverter 22 is sufficient for controlling the power, implementing the control method, the type of the switching element, etc.

Thus, the inverter 22 outputs an AC voltage corresponding to the input DC voltage, and more specifically an AC voltage that is proportional to the input voltage. For example, 1 kV AC is outputted if 1 V DC is applied, and 9 kV AC is outputted if 9 V DC is applied. In order to generate stable cold plasma, the output voltage needs to be controlled based on certain structural parameters of the plasma generator 10, such as the distance between the electrodes 16a, 16b, the material from which the electrodes are formed, the plane size and the thickness of the electrodes 16a, 16b. The AC frequency may be appropriately determined.

In some embodiments, the inverter 22 converts DC 12V to AC33.3V and transmits this voltage to the booster 24. The control method used to apply voltage to the electrodes 16a, 16b to maintain stable barrier discharge, and the amount of voltage sent to the booster, is controlled by the amplitude of the inverter output. A switching element for the separation unit 21 (described below) uses a Field Effect Transfer (FET) and can be built into the inverter. For example, in some embodiments, the Field Effect Transfer (FET) is built into the inverter, while in other embodiments the Field Effect Transfer (FET) is structurally separate from the inverter. The booster 24 uses a rate of 150× to convert the AC33.3V received from inverter up to AC 15 kV.

In some embodiments, the inverter 22 converts DC 12V to AC 20V-AC 100V and transmits this voltage to the booster 24. The booster can have a predetermined rate of 150× and be capable of converting AC 20V-AC 100V to AC 3 kV-15 kV. It may be beneficial to boost AC 33.33V to AC 5 kV for stable cold plasma generation. The composition of the gas passing through the plasma Gap 15 may affect the voltage that is relevant or required for the stable cold plasma generation. In some applications, such as disinfecting and/or sterilizing, less voltage may be necessary, such as AC 5 kV, but in other applications, such as decomposing gases, higher voltage may be necessary, such as AC 7.5 kV-15 kV. The booster 24 can be part of the power supply 21 and can be a transformer.

However, embodiments are intended to cover other voltages and voltage ranges as discussed in detail below. For example, 5 kV-10 kV may be especially beneficial, and 7.5 kV may be even more beneficial. Further, in some embodiments where ozone is a concern, the voltage levels may be beneficial at 3 kV-7 kV. However, 5 kV may be even more beneficial in this context.

Although barrier discharge is used in the plasma generator 10, discharge does not occur if the voltage between the electrodes 16a, 16b is too low. If the voltage between the electrodes 16a, 16b is too high, then it shifts to spark discharge or arc discharge, which leads to a decrease in the generation efficiency of active species by the plasma and damage of the electrodes 16a, 16b due to the concentration of discharge in a specific place.

In the present embodiments, the stable barrier discharge is maintained by controlling the AC voltage applied between the electrodes 16a, 16b. Thus, the generation of plasma can be stably and continuously performed. Further, by controlling an AC voltage applied between the electrodes, a multi-plasma gas containing active oxygen species is efficiently generated while suppressing generation of harmful ozone (O3).

Voltage levels that produce stable barrier discharge range from 3 kV to 9 kV, although 5 kV may be more advantageous. Due to electrochemical properties, the amount of ozone generated is controlled by voltage. Low voltage 5 kV, for example, generates lower ozone concentrations and the higher voltage 10 kV, for example, generates higher amounts of ozone. The applicable voltage levels may increase to allow for additional plasma generators 10 to be stacked to allow for stable plasma generation. In some embodiments where ozone is not a concern, such as for CO2 decomposition, the voltage levels that may be beneficial are 3 kV-15 kV. However, 5 kV-10 kV may be especially beneficial, and 7.5 kV may be even more beneficial. Further, in some embodiments where ozone is a concern, the voltage levels may be beneficial at 3 kV-7.5 kV. However, 5 kV may be even more beneficial.

The frequency of the electrical power supplied to the electrodes is also relevant to the plasma generation. The frequency is determined by the inverter 22 and can range from 30-60 Hz. At 50 Hz, frequency equals 20 ms and streamer discharge at 10 ms. At 60 Hz, frequency equals 16.67 ms and streamer discharge at 8.33 ms.

As discussed above, multiple stacked layers of electrodes 16a, 16b and dielectric layers 14 can be used to scale each plasma generator 10, and in fact separate plasma generators 10 can be stacked such as by being vertically aligned. The voltage used to power multiple stacked plasma generators 10 can be augmented by the booster 24 and the inverter 22. The voltage used to power multiple stacked plasma generators 10 can also be augmented by the power supply 21 or one or multiple transformers.

Thus, as discussed above, creating a stable cold plasma depends on a combination of elements, including voltage, frequency, composition of electrodes and dielectric material, structure, size, spacing, creepage distance, and process of forming electrodes. However, the selected voltage and frequency of the electrical power supplied to the electrodes 16a, 16b and the dielectric strength and relative permittivity are especially important to generate stable cold plasma.

Tables in FIG. 6 and FIG. 7 describe specific parameters of the plasma generator. Each table describes specific examples of parameters that may be found in exemplary embodiments. Each table references parameters depicted in configurations described in FIGS. 1-5. However, it is important to note that the parameters disclosed therein are provided merely for exemplary purposes and are not intended to be limiting in any way. In fact, embodiments are intended to include or otherwise cover plasma generators that include any parameters to create or facilitate the creation of stable cold plasma in a fluid. Embodiments are intended to cover any type of fluid composition in the context of the generation of ions, including but not limited to, power plant flue gas, automobile exhaust gas, chemical processing plant exhaust gas, manufacturing plant exhaust gas, and atmospheric air. Atmospheric air is defined here to be gas composed of the same relative constituents, temperatures, and pressures as the average atmospheric conditions in the location of the device, with humidity that varies naturally.

The tables shown in FIG. 6 and FIG. 7 convey the large changes in the plasma generation process that stem from minute changes to the configuration and other parameters. Each table references a single stacked layer configuration of the plasma generator 10 found in FIGS. 1-5, where there is a first electrode 16a and second electrode 16b, each with a dielectric layer 14 on the surface that faces the gap created between the two electrodes. However, the parameters in each table in FIG. 6 and FIG. 7 can be multiplied for each layer present in a particular embodiment that contains multiple or stacked plasma generating layers. The optimization of the plasma device for the formation of free radicals and other reactive species is shown to depend on minute changes in the parameters. These parameters are dependent on constants such as the space between each layered electrode plates, the thickness of the dielectric material and the dimensions of the plasma gap. Some of these constants are changed between FIG. 6 and FIG. 7 to show some of the changes to the properties of the system due to the changes in the configuration and materials of the device. Although FIG. 6 and FIG. 7 are keeping electrode material, electrode gap, dielectric material, frequency of applied voltage, and applied voltage constant, it is assumed that other constants can be held and the calculations and parameters will be adjusted in order to produce stable cold plasma.

Some parameters are well defined and described in explicit units, while other parameters are difficult to measure. Parameters that are difficult to measure are described in the tables using an arbitrary qualitative scale, or a scale used to convey qualitative changes.

FIG. 6 is a table of different parameters for a plasma generator with dielectric layers formed of borosilicate glass. Every column alternates between parameters for an exemplary small electrode configuration (6 cm×9 cm) and parameters for an exemplary large electrode configuration (30 cm×30 cm), as noted in the top row. However, as indicated previously herein, embodiments are intended to include or otherwise cover other electrode sizes.

The first parameter, starting from Row 1, is “Electrode Material”. The electrode is the circuit element that allows current to pass through its volume to its surface, but the electrons stay confined to the surface and cannot pass through the dielectric material adjoining the surface of the electrode directly opposite the incoming current. This parameter is constant through all variations in the table and between FIG. 6 and FIG. 7, with the single value of “Aluminum” but can be made of similar conductive material such as stainless steel.

Row 2 is “Electrode Gap”, which describes the distance between the surfaces of two layered electrodes in millimeters (mm). This parameter is a constant and thus remains unchanged through all variations in the table and has a single value of “6 mm.”

Row 3 is “Dielectric Layer Material”, which describes the material that constitutes the dielectric layers of the plasma generator. In this embodiment, the dielectric layer is on each stacked electrode. The dielectric material is defined to be a material that can inherently polarize in the presence of an external electric field, thus reducing the electric field within. The charges within the dielectric layer are bound and thus its purpose is to prevent any current from passing through its bulk. This then limits the amount of current that passes through the fluid between the dielectric surfaces, preventing the current from heating the fluid far above room temperature. This parameter also remains unchanged through all variations in the table and has the single value of “Borosilicate Glass”.

Row 4 is “Dielectric Thickness”, which is the thickness of each of the two dielectric layers between the electrodes in millimeters. This parameter increases as the columns progress to the right of the table. This parameter is an independent variable, and the dependent parameters depend on its value. This means that this is the only parameter that is actively being varied in the chart and all changes of other parameters are completely dependent on the changes of the independent variable “Dielectric Thickness”.

Row 5 is “Relative Permittivity”, which is the dimensionless ability of the dielectric layer to reduce an externally applied electric field within the dielectric material—in other words its ability to polarize in an external electric field. The relative permittivity of a vacuum is assumed to be 1, and all other relative permittivity values are greater than this value. This parameter depends on the “Dielectric Layer Material” row and the “Frequency” row, and thus remains unchanged through all variations in the table because the dielectric layer and frequency are constant, having the single value of “4.7”.

Row 6 is “Dielectric Strength”, which is the electric field threshold that initiates transition from insulator (i.e., dielectric layer 14) to conductor (i.e., electrode 16a, 16b) and allows significant electric current to pass through. An insulator is a material with the ability to prevent electric charge carriers from passing through at a certain electric field (i.e., dielectric layer 14), whereas a conductor is a material that can allow large amounts of charge carriers to pass through at the same voltage (i.e., electrode 16a, 16b). For high voltage conditions, a higher dielectric strength is desirable so that charge is allowed to build on or over the surfaces of the dielectric layers and does not pass through the dielectric material. If significant electric current were to pass through the dielectric layer, the purpose of the dielectric would become obsolete, because the current through the fluid in the plasma gap 15 would no longer be limited. This would cause significant heating of the fluid and power consumption would increase, vastly decreasing the efficiency of the device. This parameter depends on the material used for the “Dielectric Layer Material” row and thus remains unchanged through all variations in the table and has the single value of “30 kV/mm”.

Row 7 is “Plasma Gap”, which is the distance in millimeters between the two dielectric layers, and the region that contains the gas/fluid and in which the stable cold plasma is generated i.e., plasma gap 15. This parameter decreases as from left to right in the table due to the dielectric layer increasing in thickness.

Row 8 is “Frequency”, which is the frequency in Hertz (Hz) of the alternating current (AC) applied to the electrodes that are connected to the plasma power supply 20. This parameter remains unchanged through all variations in the table and has the single value of “60 Hz”.

Row 9 is “Applied Voltage”, which is the RMS (root-mean-square) value in kilovolts (kV) of the AC voltage applied to at least one of the electrodes connected to the plasma power supply 20. This parameter remains unchanged through all variations in the table and has the single value of “5 kV”.

Row 10 is “Streamer Discharge”, which is the amount of time during a bipolar wave pulse if plasma micro discharges form within the gas/fluid, or the time during which the gap between the dielectric layer surfaces is at the absolute value of burning voltage, which is another parameter that is described below. A bipolar wave pulse is also known as a single alternating current cycle. Micro discharges are the individual filaments that allow current to pass between the surfaces of the dielectrics and are the actual plasma forming regions in the gas/fluid. Micro discharges occur on the order of nanoseconds.

The voltage between the dielectric layer surfaces, in the plasma/fluid gap region, is known as the plasma gap voltage, which is different from the applied voltage. The plasma gap voltage is the mechanism by which the streamer discharge and other parameters vary, and the concept can be used to justify qualitative changes in those parameters. The existence of a micro discharge is dependent on the value of the plasma gap voltage at a particular moment of time: if the plasma gap voltage is too low, the micro discharges cannot exist, and if the plasma gap voltage reaches the critical value of the burning voltage, the micro discharges can form plasma. The plasma gap voltage depends on the charges built up on the electrode surfaces and on the charges built up on the dielectric layer surfaces. Once the charge buildup on the electrodes creates a plasma gap voltage during the rising portion of the AC cycle that is equal to a threshold value known as the burning voltage (described below), micro discharges carry small amounts of charge from one dielectric layer surface to the other.

The charge carried by a micro discharge creates a negative voltage that brings the plasma gap voltage below the value of the burning voltage. This process happens every time the plasma gap voltage momentarily rises above the burning voltage. There is also subtle variability in the surface charges on the surfaces of the dielectric layers, so a micro discharge in one location in the plasma gap 15 will not significantly influence the plasma gap voltage in another location in the plasma gap 15. Thus, there may be many micro discharges occurring at once that all work to keep the average plasma gap voltage at the value of the burning voltage through the existence of the micro discharges. Using an arbitrary qualitative scale, the value of the streamer discharge parameter increases from left to right on the table for small electrodes, and then increases on a separate scale for large electrodes.

However, if changing from a small electrode to a large electrode while keeping the independent variable of dielectric layer thickness constant, the value of this parameter decreases. In other words, the amount of time during which the absolute value of the average plasma gap voltage between the dielectric layers surfaces is at the value of burning voltage increases as the thickness of the dielectric layer is increased and the plasma gap distance (height if electrodes are stacked vertically) is decreased. The reason for this might be that the streamer discharge roughly follows the changes in capacitance (described below). A lower capacitance will lead to a faster buildup of charge and thus a larger plasma gap voltage. The time that the plasma gap voltage is at the burning voltage will then increase, leading to an increase in the value of this parameter.

Row 11 is “Capacitance”, which is the amount of surface charge that can be stored on the electrode plates divided by the voltage difference between the electrode plates. For multiple dielectric layers (the dielectric layers and the gas/fluid in the plasma gap, which also has dielectric properties), the following formula is used: where C is the capacitance, is the vacuum permittivity constant, A is the surface area of one of the electrode plates, is the relative permittivity of the dielectric layer, is the relative permittivity of the gas/fluid in the plasma gap, d1 is the combined thickness of both of the dielectric layers, and d2 is the plasma gap distance between the dielectric layers.

The capacitance in the context of the disclosed embodiments represents the amount of time it takes for the electrode plates to build up electric charge, which is the mechanism responsible for inducing an electric field within the plasma gap. In an AC cycle, this also represents how close the plasma gap voltage within the plasma gap is to the applied voltage. Higher values of capacitance might decrease the time and the amount of plasma in the gap. The capacitance increases if changing from a small electrode configuration to a large electrode configuration with all other parameters remaining constant. The capacitance increases then the thickness of the dielectric layer is increased, represented in the table from left to right in the table.

Row 12 is “Impedance”, which is the amount of resistance to the flow of alternating current into and out of the electrodes. The impedance inversely correlates with the induced current, with lower impedance inducing greater current. This relationship can be demonstrated by Ohm's law for AC circuits, where impedance is equal to the root mean square (RMS) value of applied voltage divided by the RMS value of induced current. In the calculation below, resistive effects are ignored and only capacitive effects are considered, because capacitive effects dominate.

In this case, the impedance is equal to the magnitude of the capacitive reactance (a well-known electrical engineering term that describes the amount of electrical energy an AC circuit can store in a capacitor), with the following formula: where Z is the impedance, f is the frequency of the applied alternating current, and C is the capacitance of the device. The impedance of the plasma generator decreases if changing from a relatively small electrode configuration to a relatively large electrode configuration. The impedance of the plasma generator increases if the thickness of the dielectric layer is increased, represented in the table from left to right in the table.

Row 13 is “Peak Reduced Electric Field”, which is the peak electric field divided by the number density of the fluid. This parameter is defined in Townsend units (Td). The approximate plasma gap voltage is calculated to be the applied voltage divided by 0.707 (in order to convert root mean square applied voltage to peak applied voltage), and thus the peak reduced electric field is the peak plasma gap voltage divided by the distance between the electrodes (i.e., 6 mm in the current embodiment), which is the plasma gap plus the thickness of both dielectric layers. The following formula is used to calculate the peak reduced electric field: where E/N is the approximate peak reduced electric field, V is the RMS applied voltage, d is the electrode distance, n_0 is the Loschmidt constant, equal to, and the factor of 10\hat{ }21 is used to convert to Townsend units.

The true plasma gap voltage is lower than the applied plasma gap voltage applied to the electrodes and changes due to capacitive effects. This parameter depends on the “Electrode Gap” row, and the “Applied Voltage” row and remains unchanged through all variations in the table because all of these parameters remain constant, having the single value of “43.82 Td”.

Row 14 is “Ignition Voltage”, which is the voltage between the electrodes that initiates the very first micro discharge in the plasma gap in the operation of the entire device. This parameter depends mainly on the surface properties of the dielectric layer material, the distance between the dielectric layers, and the composition of the gas/fluid in the plasma gap. The ignition voltage can also depend on the amount of time the device has been turned off between operations, decreasing with smaller breaks in operation. Using an arbitrary qualitative scale, the value of this parameter decreases from left to right in the table, every other column. In other words, the ignition voltage decreases if the thickness of the dielectric layer is increased.

Row 15 is “Burning Voltage”, which is the voltage between the electrodes that initiates sustained micro discharges after many AC cycles. This voltage is an asymptotic property, meaning the voltage that initiates micro discharges will lower from the ignition voltage and approach the burning voltage over many AC cycles. Using an arbitrary qualitative scale and comparing it to the “Ignition Voltage” row, the value of this parameter is always lower than that of the ignition voltage and decreases from left to right in the table, every other column. In other words, the burning voltage also decreases if the thickness of the dielectric layer is increased.

Row 16 is “Micro Discharge Number”, which is the number of micro discharges present in the plasma gap region at any one time. Because micro discharges have a filamentary volumetric shape and all are the same size, the number of micro discharges is nearly synonymous with the momentary volume of plasma present in the plasma gap region. Using an arbitrary qualitative scale, the value of this parameter increases when traveling from left to right in the table, but at separate scales for small electrodes and large electrodes.

If changing from a small electrode to a large electrode while keeping the independent variable of dielectric layer thickness constant, the value of this parameter also increases. In other words, the momentary plasma volume increases if the thickness of the dielectric layer is increased or if the surface area of the electrodes is increased. The reason for this might be related to the decrease in burning voltage; if the applied voltage remains constant, the burning voltage threshold occurs earlier in the AC cycle where the slope of the plasma gap voltage is greater. This will cause the number of micro discharges to increase in order to more quickly cancel the rising plasma gap voltage and keep it at the value of the burning voltage.

However, in some configurations, the decrease in plasma gap distance will decrease the length of each micro discharge, which might also decrease the volume of the micro discharges to a small degree. This might lead to either a smaller increase in the micro-discharge number or a decrease in micro-discharge number if increasing the thickness of the dielectric layer.

In some embodiments, there can be one or two dielectric layers between each pair of electrodes, yielding beneficial results if there are two dielectric layers between each pair of electrodes. The number of electrodes can also vary, wherein the horizontally aligned electrodes are stacked vertically with dielectric layers and fluid gaps between every stacked electrode such that first and second electrodes serve as top and bottom electrodes to adjoin one plasma gap in a stack of electrodes and dielectric layers to create multiple plasma gaps. Embodiments are intended to include or otherwise cover any number of electrodes and resulting gaps. In some embodiments, the number of electrodes can be configured to be between two and fifty-one, where the number of gaps between electrodes can be between one and fifty, yielding beneficial results with the number of fluid gaps between five and twenty, and still further at ten.

The stacking process described above changes some of the parameters in the tables. The capacitance scales linearly with the number of plasma gaps, so for an arrangement of five plasma gaps, the capacitance is equal to that of a single plasma gap multiplied by five. This increase in capacitance would clearly decrease the impedance, using the formula given in the embodiments above. This would also decrease the speed of charge buildup on the electrode plate surfaces, and thus lowering the plasma gap voltage between the plates. The lower plasma gap voltage would then decrease the amount of time that the plasma can be held at the burning voltage, decreasing the streamer discharge.

FIG. 7 is a table of different parameters for a plasma generator with dielectric layers formed of ceramic. The leftmost column identifies the parameter that is assigned to the values in the same row. Every column alternates between an exemplary small electrode configuration (6 cm×9 cm) and an exemplary large electrode configuration (30 cm×30 cm), demonstrated by the top row. However, as indicated previously herein, embodiments are intended to include or otherwise cover other electrode sizes.

The first parameter, starting from Row 1, is “Electrode Material”. The electrode is the circuit element that allows current to pass through its volume to its surface, but the electrons stay confined to the surface and cannot pass through the dielectric material adjoining the surface of the electrode directly opposite the incoming current. This parameter is constant through all variations in the table and between FIG. 6 and FIG. 7, with the single value of “Aluminum”.

Row 2 is “Electrode Gap”, which describes the distance between the surfaces of two layered electrodes in millimeters (mm). This parameter is a constant and thus remains unchanged through all variations in the table and has a single value of “6 mm”.

Row 3 is “Dielectric Layer Material”, which describes the material that constitutes the dielectric layers of the plasma generator. In this embodiment, the dielectric layer is on each stacked electrode. The dielectric material is defined to be a material that can inherently polarize in the presence of an external electric field, thus reducing the electric field within. The charges within the dielectric layer are bound and thus its purpose is to prevent any current from passing through its bulk. This then limits the amount of current that passes through the fluid between the dielectric surfaces, preventing the current from heating the fluid far above room temperature. This parameter also remains unchanged through all variations in the table and has the single value of “Ceramic”.

Row 4 is “Dielectric Thickness”, which is the thickness of each of the two dielectric layers between the electrodes in millimeters. This parameter increases as the columns progress to the right of the table. This parameter is an independent variable, and the dependent parameters depend on its value. This means that this is the only parameter that is actively being varied in the chart and all changes of other parameters are completely dependent on the changes of the independent variable “Dielectric Thickness”.

Row 5 is “Relative Permittivity”, which is the dimensionless ability of the dielectric layer to reduce an externally applied electric field within the dielectric material—in other words its ability to polarize in an external electric field. The relative permittivity of a vacuum is assumed to be 1, and all other relative permittivity values are greater than this value. This parameter depends on the “Dielectric Layer Material” row and the “Frequency” row, and thus remains unchanged through all variations in the table because the dielectric layer and frequency are constant, having the single value of “25”.

Row 6 is “Dielectric Strength”, which is the electric field threshold that initiates transition from insulator (i.e., dielectric layer 14) to conductor (i.e., electrode 16a, 16b) and allows significant electric current to pass through. An insulator is a material with the ability to prevent electric charge carriers from passing through at a certain electric field (i.e., dielectric layer 14), whereas a conductor is a material that can allow large amounts of charge carriers to pass through at the same voltage (i.e., electrode 16a, 16b). For high voltage conditions, a higher dielectric strength is desirable so that charge is allowed to build on or over the surfaces of the dielectric layers and does not pass through the dielectric material. If significant electric current were to pass through the dielectric layer, the purpose of the dielectric would become obsolete, because the current through the fluid in the plasma gap 15 would no longer be limited. This would cause significant heating of the fluid and power consumption would increase, vastly decreasing the efficiency of the device. This parameter depends on the material used for the “Dielectric Material Layer” row and thus remains unchanged through all variations in the table and has the single value of “24 kV/mm”.

Row 7 is “Plasma Gap”, which is the distance in millimeters between the two dielectric layers, and the region that contains the gas/fluid and in which the stable cold plasma is generated i.e., plasma gap 15. This parameter decreases as from left to right in the table due to the dielectric layer increasing in thickness.

Row 8 is “Frequency”, which is the frequency in Hertz (Hz) of the alternating current (AC) applied to the electrodes that are connected to the plasma power supply 20. This parameter remains unchanged through all variations in the table and has the single value of “60 Hz”.

Row 9 is “Applied Voltage”, which is the RMS (root-mean-square) value in kilovolts (kV) of the AC voltage applied to at least one of the electrodes connected to the plasma power supply 20. This parameter remains unchanged through all variations in the table and has the single value of “5 kV”.

Row 10 is “Streamer Discharge”, which is the amount of time during a bipolar wave pulse during which plasma micro discharges form within the gas/fluid, or the time during which the gap between the dielectric layer surfaces is at the absolute value of burning voltage, which is another parameter that is described below. A bipolar wave pulse is also known as a single alternating current cycle. Micro discharges are the individual filaments that allow current to pass between the surfaces of the dielectrics and are the actual plasma forming regions in the gas/fluid. Micro discharges occur on the order of nanoseconds.

The voltage between the dielectric layer surfaces, in the plasma/fluid gap region, is known as the plasma gap voltage, which is different from the applied voltage. The plasma gap voltage is the mechanism by which the streamer discharge and other parameters vary, and the concept can be used to justify qualitative changes in those parameters. The existence of a micro discharge is dependent on the value of the plasma gap voltage at a particular moment of time: if the plasma gap voltage is too low, the micro discharges cannot exist, and if the plasma gap voltage reaches the critical value of the burning voltage, the micro discharges can form plasma. The plasma gap voltage depends on the charges built up on the electrode surfaces and on the charges built up on the dielectric layer surfaces.

Once the charge buildup on the electrodes creates a plasma gap voltage during the rising portion of the AC cycle that is equal to a threshold value known as the burning voltage (described below), micro discharges carry small amounts of charge from one dielectric layer surface to the other. The charge carried by a micro discharge creates a negative voltage that brings the plasma gap voltage below the value of the burning voltage. This process happens every time the plasma gap voltage momentarily rises above the burning voltage. There is also subtle variability in the surface charges on the surfaces of the dielectric layers, so a micro discharge in one location in the plasma gap 15 will not significantly influence the plasma gap voltage in another location in the plasma gap 15. Thus, there may be many micro discharges occurring at once that all work to keep the average plasma gap voltage at the value of the burning voltage through the existence of the micro discharges.

Using an arbitrary qualitative scale, the value of the streamer discharge parameter increases from left to right on the table for small electrodes, and then increases on a separate scale for large electrodes. However, if changing from a small electrode to a large electrode while keeping the independent variable of dielectric layer thickness constant, the value of this parameter decreases. In other words, the amount of time during which the absolute value of the average plasma gap voltage between the dielectric layers surfaces is at the value of burning voltage increases as the thickness of the dielectric layer is increased and the plasma gap distance (height if electrodes are stacked vertically) is decreased. The reason for this might be that the streamer discharge roughly follows the changes in capacitance (described below). A lower capacitance will lead to a faster buildup of charge and thus a larger plasma gap voltage. The time that the plasma gap voltage is at the burning voltage will then increase, leading to an increase in the value of this parameter.

Row 11 is “Capacitance”, which is the amount of surface charge that can be stored on the electrode plates divided by the voltage difference between the electrode plates. For multiple dielectric layers (the dielectric layers and the gas/fluid in the plasma gap, which also has dielectric properties), the following formula is used: C=ϵA κ1κ2/(κ2d1+κ1d2) where C is the capacitance, ϵ is the vacuum permittivity constant, A is the surface area of one of the electrode plates, κ1 is the relative permittivity of the dielectric layer, κ2 is the relative permittivity of the gas/fluid in the plasma gap, d1 is the combined thickness of both dielectric layers, and d2 is the plasma gap distance between the dielectric layers.

The capacitance in the context of the disclosed embodiments represents the amount of time it takes for the electrode plates to build up electric charge, which is the mechanism responsible for inducing an electric field within the plasma gap. In an AC cycle, this also represents how close the plasma gap voltage within the plasma gap is to the applied voltage. Higher values of capacitance might decrease the time and the amount of plasma in the gap. The capacitance increases if changing from a small electrode configuration to a large electrode configuration with all other parameters remaining constant. The capacitance increases when the thickness of the dielectric layer is increased, represented in the table from left to right in the table.

Row 12 is “Impedance”, which is the amount of resistance to the flow of alternating current into and out of the electrodes. The impedance inversely correlates with the induced current, with lower impedance inducing greater current. This relationship can be demonstrated by Ohm's law for AC circuits, where impedance is equal to the root mean square (RMS) value of applied voltage divided by the RMS value of induced current. In the calculation below, resistive effects are ignored, and only capacitive effects are considered, because capacitive effects dominate.

In this case, the impedance is equal to the magnitude of the capacitive reactance (a well-known electrical engineering term that describes the amount of electrical energy an AC circuit can store in a capacitor), with the following formula Z=½πfC where Z is the impedance, f is the frequency of the applied alternating current, and C is the capacitance of the device. The impedance of the plasma generator decreases if changing from a relatively small electrode configuration to a relatively large electrode configuration. The impedance of the plasma generator decreases if the thickness of the dielectric layer is increased, represented in the table from left to right in the table.

Row 13 is “Peak Reduced Electric Field”, which is the peak electric field divided by the number density of the fluid. This parameter is defined in Townsend units (Td). The approximate plasma gap voltage is calculated to be the applied voltage divided by 0.707 (in order to convert root mean square voltage to peak voltage), and thus the peak electric field is the plasma gap voltage divided by the distance between the electrodes (i.e., 6 mm in the current embodiment), which is the plasma gap plus the thickness of both dielectric layers. The following formula is used to calculate the peak reduced electric field: E/N=(V)/((0.707) d [symbol] n [symbol] _0)×(1 [symbol] 0 [symbol] \hat{ }21 [symbol] [symbol] V [symbol] \hat{ }(−1)m [symbol] \hat{ }(−2)) where E/N is the approximate peak reduced electric field, V is the RMS applied voltage, d is the electrode distance, [symbol] n [symbol] _0 is the Loschmidt constant, equal to 2.7×1 [symbol] 0 [symbol] \hat{ }25 [symbol] m [symbol] \hat{ }(−3), and the factor of 1 [symbol] 0 [symbol] \hat{ }21 is used to convert to Townsend units.

The true plasma gap voltage is lower than the applied voltage that is applied to the electrodes and changes due to capacitive effects. This parameter depends on the “Electrode Gap” row, and the “Applied Voltage” row and remains unchanged through all variations in the table because all of these parameters remain constant, having the single value of “43.82 Td”.

Row 14 is “Ignition Voltage”, which is the voltage between the electrodes that initiates the very first micro discharge in the plasma gap in the operation of the entire device. This parameter depends mainly on the surface properties of the dielectric layer material, the distance between the dielectric layers, and the composition of the gas/fluid in the plasma gap. The ignition voltage can also depend on the amount of time the device has been turned off between operations, decreasing with smaller breaks in operation. Using an arbitrary qualitative scale, the value of this parameter decreases from left to right in the table, every other column. In other words, the ignition voltage decreases if the thickness of the dielectric layer is increased.

Row 15 is “Burning Voltage”, which is the voltage between the electrodes that initiates sustained micro discharges after many AC cycles. This voltage is an asymptotic property, meaning the voltage that initiates micro discharges will lower from the ignition voltage and approach the burning voltage over many AC cycles. Using an arbitrary qualitative scale and comparing it to the “Ignition Voltage” row, the value of this parameter is always lower than that of the ignition voltage and decreases from left to right in the table, every other column. In other words, the burning voltage also decreases if the thickness of the dielectric layer is increased.

Row 16 is “Micro Discharge Number”, which is the number of micro discharges present in the plasma gap region at any one time. Because micro discharges have a filamentary volumetric shape and all are the same size, the number of micro discharges is nearly synonymous with the momentary volume of plasma present in the plasma gap region. Using an arbitrary qualitative scale, the value of this parameter increases when traveling from left to right in the table, but at separate scales for small electrodes and large electrodes. If changing from a small electrode to a large electrode while keeping the independent variable of dielectric layer thickness constant, the value of this parameter also increases. In other words, the momentary plasma volume increases if the thickness of the dielectric layer is increased or if the surface area of the electrodes is increased.

The reason for this might be related to the decrease in burning voltage; if the applied voltage remains constant, the burning voltage threshold occurs earlier in the AC cycle where the slope of the plasma gap voltage is greater. This will cause the number of micro discharges to increase in order to cancel the rising plasma gap voltage and keep it at the value of the burning voltage more quickly. However, in some configurations, the decrease in plasma gap distance will decrease the length of each micro discharge, which might also decrease the volume of the micro discharges to a small degree. This might lead to either a smaller increase in the micro-discharge number or a decrease in micro-discharge number if increasing the thickness of the dielectric layer.

In some embodiments, there can be one or two dielectric layers between each pair of electrodes, yielding beneficial results if there are two dielectric layers between each pair of electrodes. The number of electrodes can also vary, wherein the horizontally aligned electrodes are stacked vertically with dielectric layers and fluid gaps between every stacked electrode such that first and second electrodes serve as top and bottom electrodes to adjoin one plasma gap in a stack of electrodes and dielectric layers to create multiple plasma gaps. In some embodiments the number of electrodes can be configured to be between two and fifty-one, where the number of gaps between electrodes can be between one and fifty, yielding beneficial results with the number of fluid gaps between five and twenty, and still further at ten. However, embodiments are intended to include or otherwise cover any number of electrodes and/or gaps.

The stacking process described above changes some of the parameters in the tables. The capacitance scales linearly with the number of plasma gaps, so for an arrangement of five plasma gaps, the capacitance is equal to that of a single plasma gap multiplied by five. This increase in capacitance would clearly decrease the impedance, using the formula given in the embodiments above. This would also decrease the speed of charge buildup on the electrode plate surfaces, and thus lower the plasma gap voltage between the plates. The lower plasma gap voltage would then decrease the amount of time that the plasma can be held at the burning voltage, decreasing the streamer discharge.

As indicated above, the parameters provided in the tables of FIGS. 6 and 7 are provided for exemplary purposes, and it is intended that other embodiments include other parameters.

In some embodiments, the plasma generator 10 may be assisted in its function of sterilization by an ultrasonic oscillator, which is a device that emits ultrasonic waves and serves to support cleaning of air by damaging structures such as bacteria in the air or by promoting mixing of various active species generated in the atmospheric pressure low-temperature plasma by the plasma generator 10.

In some embodiments, the plasma generator 10 may also be assisted by an ultraviolet emitter, which emits ultraviolet light and can help to sterilize the air by destroying bacteria or virus. The ultraviolet light can also help to ionize the air, possibly reducing the amount of applied voltage required.

### E. Decreased Ozone Production

The chemical products of the plasma, including the relative productions of ozone versus other reactive oxygen species or free radicals, are determined at least in part by the properties of the individual micro discharges. Thus, each individual micro discharge can be regarded as a miniature non-equilibrium plasma chemical reactor. The micro discharge characteristics can be tailored for a given application, such as decreasing the relative production of ozone compared to hydroxyl radicals, by making use of the fluid properties, adjusting the electrode design, or changing the dielectric material thereby changing the properties of the dielectric layer. The properties of each micro discharge are notably independent of the electrode area or the applied voltage, which only serve to change the number of micro discharges present in the fluid gap. Thus, individual micro discharge properties are not altered during up-scaling.

From the plasma chemistry perspective, a significant or dominant characteristic of DBD is the dynamics of the electron energy distribution function (EEDF) in the micro discharge. The EEDF changes based on properties such as the value of the relative permittivity of the dielectric layer, the thickness of the dielectric layer, the electron work function of the dielectric surface, the roughness and porousness of the dielectric surface, the thickness of the fluid gap, the molecular composition of the fluid in the fluid gap, the pressure and temperature of the fluid in the fluid gap, and more. The exact proportionalities depend on the design and arrangement of the DBD.

In order to decrease the relative production of ozone compared to hydroxyl radicals, the above properties can be manipulated to increase the tail end of the EEDF (the higher energy end) above the ionization energy of fluid, including water, which leads to production of hydroxyls, but below the ionization energy of oxygen gas, which leads to ozone production. Thus, the choice of dielectric material properties, and the thickness of the dielectric layer combined with the properties of the ambient air, including water, to decrease ozone production while still producing hydroxyls for sterilization. Because ambient air conditions are held constant through all configurations of the plasma generator (for the embodiments that use ambient air as the fluid), the choice of dielectric properties can thus be selected for the ambient air conditions to achieve an optimum or enhanced EEDF.

In some embodiments, the EEDF is tailored to produce less ozone by configuring the dielectric layer with a dielectric material that has an average surface roughness between 0 nm and 800 nm. Depending on the other stated properties that affect the EEDF, beneficial results may be yielded if a low surface roughness is desirable, between 0 and 100 nm. Beneficial results may also be yielded if a high surface roughness is desirable, between 100 and 800 nm. Low surface roughness can be used to increase the tail end of the EEDF, and high surface roughness can accomplish the opposite. This is achieved because higher surface roughness can make the electric field near the dielectric surface very nonuniform, decreasing the burning voltage, and lowering the total electric field energy transferred to each micro discharge.

In some embodiments, the EEDF is tailored to produce less ozone by configuring the dielectric layer with a dielectric material that has a relative permittivity between 2 and 500, and a dielectric layer thickness between 0 mm and 3 mm. If the relative permittivity is on the lower end, between 2 and 15, beneficial results for the dielectric thickness are yielded between 1 mm and 3 mm, with even better results at 2 mm. If the relative permittivity is in the middle of each end, between 15 and 100, beneficial results for the dielectric thickness are yielded between 0 mm and 2 mm, with even better results at 1 mm. If the relative permittivity is on the higher end, between 100 and 500, beneficial results for the dielectric thickness are yielded between 0 mm and 5 mm, with even better results at 2 mm. These combinations of relative permittivity and dielectric layer thickness affect the specific capacitance of the barrier C_d/A, which determines the amount of charge transferred in each micro discharge and, furthermore, the energy dissipated to the electrons in each micro discharge. These properties allow the tail end of the EEDF to be between that of the ionization energies of water and diatomic oxygen gas.

Plasma Activated Water

### A. Applications

As discussed above, plasma can be used to deactivate or destroy harmful microorganisms and other harmful organic or particulate matter. Various mechanisms are used to deactivate the harmful substances. In some embodiments, the plasma forms reactive species such as ROS and RNS that react chemically with molecules in the harmful substances. In these embodiments, the formed ROS and RNS have short lifetimes and can sometimes only be effective when the harmful substances are spatially close to the plasma forming region. It may therefore be beneficial to capture the ROS and RNS in order to extend their lifetimes so that they can be used in a wider variety of applications.

The lifetimes of most ROS and RNS in atmospheric air are on the order of one second or less. In some embodiments, the ROS and RNS are suspended in water after formation, increasing the lifetimes to the order of one hour, which is a several-thousand-fold increase. When the water is applied to various substances, the reactive species suspended in the water can react with harmful substances to destroy or deactivate them.

The water with suspended reactive species can be used in many applications. For example, applications include but are not limited to environment (e.g., drinking water treatment and sterilization, waste water treatment), food (e.g., food sterilization, food treatment), PFAS (perfluoroalkyl and polyfluoroalkyl substances) and PFCs (perfluorinated chemicals) removal, agriculture (e.g., seed germination, bactericidal and fungicidal agents for crops and animals, fertilizer, agricultural waste treatment, fish farm), nanoparticle synthesis (e.g., catalyst production, fuel cells, battery electrodes, magnetic materials, nanofluids), medical (e.g., hospital hygiene, antifungal treatment, dental care, skin disease, wound care, body sanitizer solutions), consumer products (e.g., washing machine, shower heads), military (e.g., biowater, biochemical defense) and cosmetic, among others.

In some embodiments, the untreated water itself may have suspended harmful substances. It may therefore be beneficial to mix the reactive species with the untreated water as a final application in order to clean or sterilize the water. Since the reactive species have a limited lifespan within the water, after a period of time the water will be cleaned or sterilized without the presence of any reactive species.

Though water is used as an example in this and the following sections, embodiments are intended to cover any liquid that can be used to effectively mix and contain reactive species.

### B. Overview of the Plasma Activated Liquid

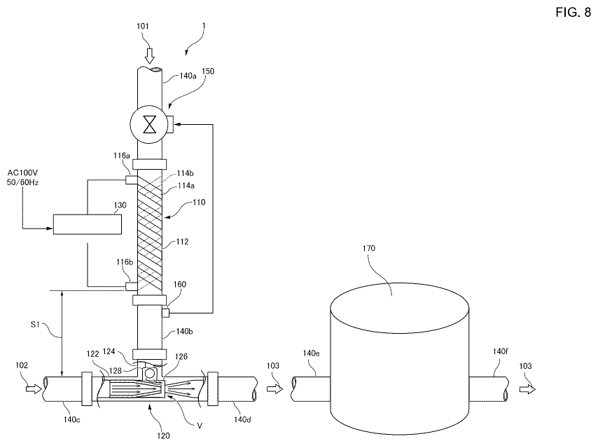

First, an overall configuration of a liquid processing apparatus according to an embodiment of the present technology will be described. FIG. 8 schematically shows the overall configuration of the liquid processing apparatus 1 according to the present embodiment. As shown in FIG. 8, the liquid processing apparatus 1 includes a plasma generating apparatus 110 for generating atmospheric pressure low-temperature plasma, an aspirator 120 as a gas suction means, a power supply apparatus 130 for supplying power to the plasma generating apparatus 110, and a flow rate adjusting apparatus 150 for adjusting the flow rate of gas supplied to the plasma generating apparatus 110.

In FIG. 8, a gas 101 to be subjected to plasma processing is supplied into the plasma generating apparatus 110 through a pipe 140a connected to an upper opening thereof. A flow rate adjusting device 150 is provided in the middle of the pipe 140a to adjust the flow rate of the gas supplied from the pipe 140a to the plasma generating apparatus 110. The flow rate adjusting device 150 has a valve device for adjusting the flow rate of the gas passing through the pipe 140a, and the opening of the valve device is adjusted based on a detection value from a pressure sensor 160 described later.

The gas treated by the atmospheric pressure low-temperature plasma in the plasma generating apparatus 110 is sucked into the intake port 124 of the aspirator 120 connected to the other end of the pipe 140b through the pipe 140b connected to the lower opening of the plasma generating apparatus 110. In some embodiments, the pipe 140b may be provided with a pressure sensor 160 for measuring the pressure in the pipe 140b. The pressure sensor 160 transmits the measured value of the pressure in the pipe 140b to the flow rate adjusting device 150 provided in the pipe 140a. The flow rate adjusting device 150 adjusts the opening of the valve device so that the inside of the pipe 140b becomes a predetermined negative pressure state lower than the atmospheric pressure based on the pressure measured value in the pipe 140b received from the pressure sensor 160. It may be beneficial to base the predetermined value of the pressure in the pipe 140b on the desired efficiency of the plasma generator 110 described below.

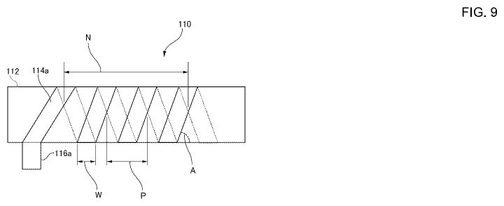

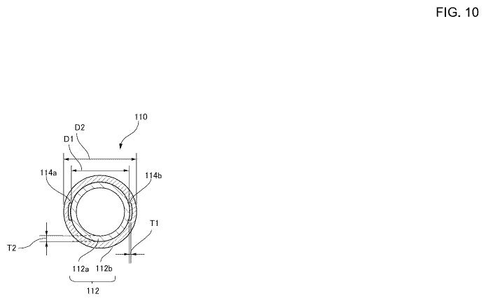

The plasma generating apparatus 110 has a function of generating atmospheric pressure low-temperature plasma in a cylindrical space formed of a hollow cylindrical member, and decomposing gas passing through the cylindrical space to generate various active species. The gas to be treated can be suitably selected according to the kind of active species generated by the plasma treatment, though a common and easy yet effective alternative can be atmospheric air. FIG. 9 is a schematic side view of the plasma generating apparatus 110, and FIG. 10 is a schematic cross-sectional view of the plasma generating apparatus 110.

The plasma generating apparatus 110 is provided with a hollow cylindrical main body 112, a first electrode 114a and a second electrode 114b each formed of a belt-like conductive material sealed onto the main body 112, and electrode terminals 116a, 116b provided at one end of the first electrode 114a and the second electrode 114b, respectively.

The body part 112 can be an insulating cylindrical member formed from a dielectric material in a hollow cylindrical shape, and defines a cylindrical space in which near-atmospheric pressure low-temperature plasma is generated, in the inside. In some embodiments, it may be beneficial to operate the plasma generator 110 at a pressure slightly lower than atmospheric pressure, resulting in an increase in efficiency and a decrease in required applied voltage. In this embodiment, the body portion 112 is formed by providing an outer layer 112b made of a resin material on the outer periphery of the tube 112a (as shown in FIG. 10). The material of the inner tube 112a can be chosen based on its dielectric constant, with good results yielded for a dielectric constant between 2 and 50 and better results between 4 and 10 and even better results at 4.6. An example of a material chosen with a proper dielectric constant for the inner tube 112a can be Tempax glass. The material of the resin layer 112b can be an epoxy, glue, tape, or any known related art that can be used for the purpose of electrical insulation. The first electrode 114a and the second electrode 114b are each formed of a thin strip conductive material. The first electrode 114a and the second electrode 114b are preferably formed of, for example, a copper foil tape, but are not limited thereto.

As illustrated in FIGS. 8 to 10, the first electrode 114a and the second electrode 114b are helically shaped so as to rotate around the central axis of the main body 112, and the helically shaped first electrode 114a and the helically shaped second electrode 114b are further arranged so as to form a double helix around the central axis. In some embodiments, a double helix shape may be a favorable electrode geometry compared to other electrode geometries, for example two opposing straight strips, since a double helix set of electrodes controls temperature effectively. As shown in FIG. 10, the first electrode 114a and the second electrode 114b are at least partly surrounded with a resin material forming an outer layer 112b in a state of being wound around the outer peripheral surface of the dielectric tube 112a.

In some embodiments, a double helix electrode geometry is chosen in order to allow for a more uniform temperature of the dielectric tube 112a. For example, if a straight electrode geometry is used, where both electrodes are straight strips on opposing sides of the dielectric tube 112a, the temperature of the dielectric tube 112a would be high in the regions just under the electrode with a high temperature gradient to the neighboring regions, which may lower the life of the component. Twisting the electrodes into a helical shape may help to spread the heat across the dielectric tube 112a, leading to lower temperature gradients and an increase to its component life.

As illustrated in FIG. 9, in the present embodiment, a W band conductor, such as copper, forming the electrodes 114a and 114b is wound in a helix shape N times at a winding pitch P. Each electrode will make the same angle A, called the helix angle, with the direction of the main body 112. Referring to FIG. 10, each of the electrodes 114a, 114b has a thickness T1 and is sealed in an outer layer 112b constituting the main body 112. Therefore, the first electrode 114a and the second electrode 114b are arranged to face each other across the dielectric tube 112a of the main body 112 across the cylindrical space in a double helix shape. In some embodiments, however, the two helical electrodes are separated by a phase shift other than 180 degrees, so that the two electrodes are not always directly opposite each other across the dielectric tube 112a.

When a high voltage and a predetermined frequency are applied between the first electrode 114a and the second electrode 114b by a power supply device 130 to be described later, dielectric barrier discharge occurs between the first electrode 114a and the second electrode 114b, and atmospheric pressure low-temperature plasma is generated in the cylindrical space surrounded by the main body 112. In the cylindrical space of the plasma generating apparatus 110, the generated atmospheric pressure low-temperature plasma acts on air and water vapor to generate active species including various radicals such as, for example, singlet oxygen (1 O2), ozone (O3), hydroxyl radical (OH), superoxide anion radical (O2-), hydroperoxy radical (HO2), and hydrogen peroxide (H2O2), as is known. The gas containing these active species is supplied to the aspirator 120 through a pipe 140b connected to the downstream side of the plasma generating apparatus 110.

As shown in FIG. 8, a distance S1 is secured between the electrode terminal 116b of the plasma generating apparatus 110 and the intake port 124 of the aspirator 120 by the pipe 140b. With this distance, it is possible to prevent unexpected events such as leakage from the electrode of the plasma generating apparatus 110 to the liquid to be processed, and the safety of the liquid processing apparatus 1 is secured. The distance S1 may be determined based on specifications of high voltages applied to the electrode terminals 116a and 116b of the plasma generating apparatus 110.

The power supply device 130 has the function of applying a high voltage between the first electrode 114a and the second electrode 114b of the plasma generator 110. In the present embodiment, the power supply device 130 includes a power conversion device for converting the input of the commercial power supply to a high voltage to be applied between the electrodes with 100 V AC and 50/60 Hz as the input. The power converter includes, for example, a transformer, an AC/DC converter, or a combination of an AC/DC converter and a DC/AC inverter so that a DC or AC high voltage can be applied between the first electrode 114a and the second electrode 114b. Any boost or switching circuit can be used to generate the high voltage. By way of example, the power supply 130 is configured to be capable of applying an appropriate high voltage between the first electrode 114a and the second electrode 114b. Specifically, the output voltage may be adjusted according to parameters such as the distance between the electrodes, the material of the electrodes, the plane size, and the thickness. The voltage to be applied may be either DC or AC, and its frequency may be appropriately determined. The voltage waveform may also be an appropriate waveform such as a sine wave. In some embodiments, good results were obtained when the applied voltage signal was a sine wave with an amplitude between 5 kV and 30 kV and better results at 21.2 kV, or 15 kV RMS. Good results for the AC frequency were obtained between 10 Hz and 200 Hz, with better results between 30 Hz and 90 Hz, with even better results at 50 Hz.

The aspirator 120 is an apparatus for mixing the gas processed by the plasma generating apparatus 110 with a liquid 102 to be processed by the liquid processing apparatus 1 of the present embodiment. In some embodiments, the aspirator 120 may also have the function of only permitting a fluid to flow in a specified direction. As shown in FIG. 8, the aspirator 120 includes a tubular body portion 122 for circulating a liquid 102 to be processed, which is also an operating fluid for causing a decompression action, and an air intake 124 protruding from a side portion of the tubular body portion 122. Pipes 140c and 140d are respectively connected to both end openings of the tubular body part 122, and the liquid 102 to be processed flows into the aspirator 120 from the pipe 140c side, and the resulting treated fluid 103 flows out to the pipe 140d side through a venturi V provided in the tubular body part 122. A pipe 140b is connected to the opening of the intake port 124, and is configured so that the gas exiting the plasma generating apparatus 110 is introduced into the aspirator 120. As is well known, the aspirator 120 acts to depressurize the space around the flow by taking advantage of the increase in dynamic pressure due to the acceleration of the fluid passing through the venturi V.

In the aspirator 120 shown in FIG. 8, when the liquid 102 to be processed passes through the venturi V, the space communicating with the intake port 124 is reduced in pressure, whereby the treated gas processed by the plasma generating apparatus 110 from the pipe 140b is sucked into the mixing chamber 126 in the aspirator 120 and mixed with the liquid 102 to be processed flowing through the venturi V. A check valve 128 is provided between the mixing chamber 126 and the pipe 140b to prevent the liquid to be processed from flowing into the pipe 140b. Further, since the inside of the main body portion 112 of the plasma generating apparatus 110 is also depressurized by the suction action of the aspirator 120, an effect that plasma is easily generated between electrodes in the cylindrical space in the main body portion 112 can be obtained.

The operation of the liquid processing apparatus 1 according to the present embodiment having the configuration described with reference to FIGS. 8 to 10 will now be described. Note that the order of description is for convenience only, and it is not necessary to operate the apparatus in the order of description.

First, the power supply device 130 is operated, and a high voltage is applied between the first electrode 114a and the second electrode 114b of the plasma generating apparatus 110 to generate a dielectric barrier discharge between both electrodes. By this dielectric barrier discharge, near-atmospheric pressure plasma-treated low-temperature plasma is generated in the cylindrical space of the main body 112.

Second, a liquid to be treated is made to flow into an aspirator 120 from a pipe 140c. When the liquid to be treated passes through the venturi V of the aspirator 120, the inside of the mixing chamber 126 of the aspirator 120 is depressurized, and treated gas is introduced into the mixing chamber 126 through a pipe 140b connected to the intake port 124.

Third, the treated gas introduced into the mixing chamber 126 was processed by the plasma generating apparatus 110 and contains various active species. The plasma-treated gas is mixed with the liquid in the mixing chamber 126. At this time, by adjusting the flow rate of the gas supplied from the pipe 140a to the plasma generator 110 and the aspirator 120 by the flow rate adjusting device 150, the gas can be mixed into the liquid passing through the aspirator 120 by the natural production of bubbles in the aspirator 120. By mixing the gas when the liquid is in such a bubbling state, various active species contained in the plasma-treated gas can be efficiently introduced into the liquid. The size of the bubbles can be changed by the structure of the aspirator 120, with good results yielded with smaller bubbles. Thus, in some embodiments, it may be beneficial to design the aspirator 120 to form bubbles that are as small as possible in order to increase the efficiency of mixing. The treated liquid 103 flowing out from the aspirator 120 through the pipe 140d is a relevant mixture of various reactive species and gas introduced into the liquid processing apparatus 1. Liquids containing the active species can be used in a variety of applications including sterilization, removal of various viruses, and inactivation.

In some embodiments, the treated fluid 103 can be transferred to a holding tank 170 via a pipe 140e. In some other embodiments, pipe 140e can transfer the treated fluid 103 to a different component. The pipe 140e can also transfer the treated fluid 103 directly to its application to a material. For example, the pipe 140e can transfer the treated fluid 103 to a set of sprinklers that spray the treated fluid 103 onto an arrangement of agricultural items. In some embodiments, a pipe 140f can transfer the treated fluid 103 from the holding tank 170 to its application. In some other embodiments, the holding tank 170 can connect to the pipe 140c to allow the treated fluid 103 to pass through the aspirator 120 multiple times. This process is referred to as plasma reactivation and has been shown to be effective.

In some embodiments, the liquid processing apparatus 1 can be connected in series to multiple liquid processing apparatuses 1 in order to increase the flow capacity or to increase the concentration of reactive species within the treated fluid 103. In this case, the pipe 140d from one liquid processing apparatus 1 would connect to the pipe 140c of the subsequent liquid processing apparatus 1. It may be beneficial to include a space between the plasma generators 110 of each liquid processing apparatus 1 that measures at least 10 cm in order to prevent electrical arcing between the plasma generators 110 or other forms of electrical interference. It may also be beneficial to include a space between the aspirators 120 of liquid processing apparatuses 1 connected in series to prevent the bubbles of the first aspirator 120 from entering the second aspirator 120 as this may decrease the efficiency. In some embodiments, the liquid processing apparatus 1 can be connected in parallel to multiple liquid processing apparatuses 1 in order to increase the flow capacity. In some embodiments, a single liquid processing apparatus 1 may be designed to allow a maximum flowrate of 8-10 L/min of untreated fluid 102 for processing, but embodiments are intended to cover any flowrate as the reactive species concentration may be chosen for a specific application. For example, a liquid processing apparatus 1 operating at a certain set of parameters and with a specific flow rate may result in a specific concentration of reactive species in the treated fluid 103, but the concentration of reactive species in the treated fluid 103 may decrease if the operating parameters are unchanged and the flow rate is increased. In this way, some embodiments may choose a specific flow rate to correspond with a specific concentration of reactive species in the treated fluid 103. In some embodiments, it may be beneficial to increase the size of the plasma generator 110 or to connect multiple liquid processing apparatuses together to increase the maximum flow rate for a chosen application.

The dimensions of the plasma generator 110 will now be described according to FIGS. 9 and 10. It should be noted that FIG. 9 shows only a single electrode 114a in order to illustrate some of the parameters more easily, though the plasma generator 110 includes both electrodes 114a and 114b. The first and second electrodes 114a, 114b, can be formed from a foil strip of thickness T1 with beneficial results yielded between 0.01 mm and 0.5 mm with better results at 0.1 mm. The foil strip width W yields good results between 1 mm and 5 mm with better results at 2 mm. The length of the foil strip depends on the number of windings N and the pitch P. Good results for the number of windings N are yielded between 3 and 15 times, with better results between 4 and 8 times, with even better results at 6 times. Good results for the pitch P are yielded between 4 mm and 15 mm with better results between 7 mm and 12 mm with even better results at 10 mm. The length of the inner tube 112a could be any length to accommodate the electrodes, with good results at 120 mm long. The outer layer 112b is only necessary to cover the electrodes 114a, 114b, thus good results were yielded at an outer layer 112b length of 90 mm. The outer diameter D1 of the dielectric tube 112a yields good results between 2 mm and 20 mm with better results between 3 mm and 8 mm and even better results at 4 mm. The wall thickness T2 of the dielectric tube 112a yields good results between 0.1 mm and 4 mm, with better results between 0.6 mm and 1 mm and even better results at 0.8 mm. In some embodiments, the outer layer 112b may be necessary to hold the electrodes in place, to prevent discharges between the electrodes outside of the dielectric tube 112a, to insulate the electrodes from the surrounding environment, or to accomplish a mixture of these tasks. The success of these conditions is dependent on the wall thickness of the outer layer 112b, which is calculated by the expression (D2−D1)/2, where D2 is the outer diameter of the outer layer 112b. The first of these conditions can be achieved with good results at a wall thickness of 0.1 mm. The second of these conditions can be achieved with good results at a wall thickness of greater than 0.1 mm, so that the electrodes are covered. The third of these conditions can be achieved with good results at a wall thickness of 2.5 mm. Therefore, if all conditions are to be satisfied, desirable results are achieved at a wall thickness of 2.5 mm or greater. However, in some embodiments, not all conditions need to be satisfied, and thus desirable results can still be achieved with a wall thickness less than 2.5 mm.

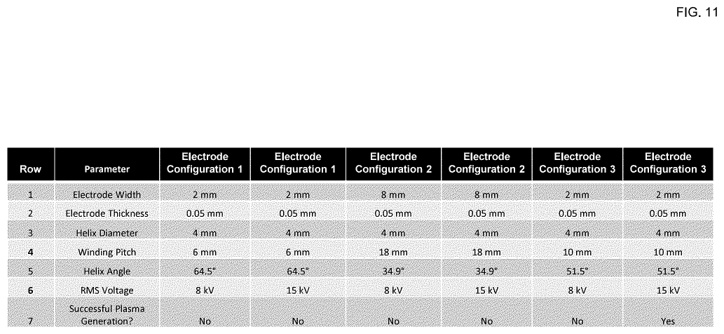

FIG. 11 describes how varying the different electrode parameters of the plasma generator 110 can lead to successful plasma generation within the dielectric tube 112a. There are three exemplary electrode configurations shown in the figure, although there are an infinite number of possible variations. The three exemplary configurations are intended to give a sample of the infinite landscape of configurations. Each of the electrode configurations is subjected to two applied voltages. Only one electrode configuration at one applied voltage generates a successful plasma within the dielectric tube 112a. The parameters of FIG. 11 will now be described in more detail below.

Row 1 is the value of the width W of the electrode strips, which is described in FIG. 9. Two tested electrode configurations have a width of 2 mm while one has a width of 8 mm. Row 2 is the value of the thickness of the electrode strips, which is described by T1 in FIG. 10. All three exemplary electrode configurations in FIG. 11 have a thickness of 0.05 mm. Row 3 is the helix diameter of each electrode in the electrode configuration. Since each electrode is wound around the dielectric tube 112a in a helical shape, the helix diameter in this case is the outer diameter of the dielectric tube 112a, which is described by the distance D1 in FIG. 10. The value of the helix diameter is 4 mm for all three exemplary electrode configurations. Row 4 is the winding pitch of each electrode, which is described by the distance P in FIG. 9. All three exemplary electrode configurations have different winding pitch, with one configuration at 6 mm, another configuration at 18 mm, and another configuration at 10 mm. To provide a more visual measure of the winding pitch, Row 5 shows the helix angle of each configuration, which is described by the helix angle A in FIG. 9. The helix angle can be calculated by the expression arctan [symbol] ((π*D1)/P). The helix angle of electrode configuration 1 is 64.5°. The helix angle of electrode configuration 2 is 34.9°. The helix angle of electrode configuration 3 is 51.5°. Row 6 is the RMS voltage of the applied voltage signal to the electrodes. Each electrode configuration is subjected to an applied voltage of 8 kV in one example and 15 kV in another. Row 7 shows whether the given electrode configuration with the given applied voltage will successfully generate a plasma within the dielectric tube 112a.

Electrode configuration 1, with an electrode width of 2 mm, an electrode thickness of 0.05 mm, a helix diameter of 4 mm, a winding pitch of 6 mm, and a helix angle of 64.5° was unsuccessful at generating a plasma within the dielectric tube 112a at an RMS voltage of both 8 kV and 15 kV. With this configuration, some plasma was generated inside the dielectric tube 112a, but also an undesirable discharge formed between the electrodes both inside and outside of the outer layer 112b. Electrode configuration 2, with an electrode width of 8 mm, an electrode thickness of 0.05 mm, a helix diameter of 4 mm, a winding pitch of 18 mm, and a helix angle of 34.9° was also unsuccessful at generating a plasma within the dielectric tube 112a at an RMS voltage of both 8 kV and 15 kV. Again, with this configuration, some plasma generated inside the dielectric tube 112a, but also a discharge formed between the electrodes both inside and outside of the outer layer 112b. Electrode configuration 3, with an electrode width of 2 mm, an electrode thickness of 0.05 mm, a helix diameter of 4 mm, a winding pitch of 10 mm, and a helix angle of 51.5° was unsuccessful at generating a plasma within the dielectric tube 112a at an RMS voltage of both 8 kV. However, once the applied RMS voltage was increased above 8 kV and to a value of 15 kV, a plasma successfully formed within the dielectric tube 112a with no discharge outside of the outer layer 112b.

In some embodiments, the plasma generator 110 may be assisted in its function of sterilization by an ultrasonic oscillator (not pictured), which is a device that emits ultrasonic waves and serves to support cleaning of air by damaging structures such as bacteria in the air or by promoting mixing of various active species generated in the atmospheric pressure low-temperature plasma by the plasma generator 110.

In some embodiments, the plasma generator 110 may also be assisted by an ultraviolet emitter (not pictured), which emits ultraviolet light and can help to sterilize the air by destroying bacteria or virus. The ultraviolet light can also help to ionize the air, possibly reducing the amount of applied voltage required.

It should be noted that the above-mentioned ultrasonic oscillator and ultraviolet emitter can be added to all embodiments of the liquid processing apparatus 1 as well as embodiments of the liquid processing apparatus 2 and the liquid processing apparatus 3.

### C. Variation of Embodiments

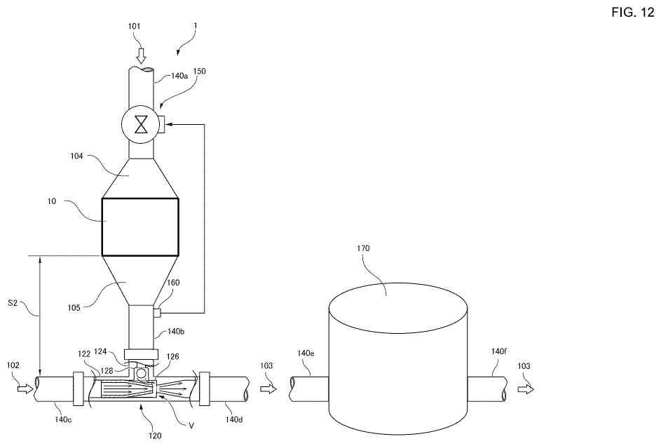

FIG. 12 shows a different exemplary configuration of the plasma activated liquid device. Instead of the plasma generator 110 described above, the configuration shown in FIG. 12 utilizes the plasma generator 10 described in FIG. 1-8.

As before, the gas 101 flows into flow rate device 150 from the pipe 140a. The gas flows out of the flow rate device 150 at a predetermined rate, that is in part decided by the value given by a pressure sensor 160, and into the entry flow adapter 104. The entry flow adapter 104 adjusts the cross-sectional flow of gas 101 to the size of the plasma generator 10. Inside the plasma generator 10, the gas 101 is ionized and turned into a plasma, where various reactive species may be formed. The treated gas flows out of the plasma generator 10 and through the exit flow adapter 105, where the cross-sectional flow of gas is again adjusted, this time to the size of the pipe 140b.

The treated gas 101 then flows into the aspirator 120, where it is mixed with an untreated fluid 102, entering the aspirator 120 through a pipe 140c, resulting in a treated fluid 103 flowing out of the aspirator 120 through a pipe 140d. A distance S2 is secured between the bottom of the plasma generator 10 and the intake port 124 of the aspirator 120 by the pipe 140b. With this distance, it is possible to prevent unexpected events such as leakage from the electrode of the plasma generating apparatus 10 to the liquid to be processed, and the safety of the liquid processing apparatus 1 is secured. The distance S2 may be determined based on specifications of high voltages applied to the electrodes of the plasma generator 10.

In some embodiments, the treated fluid 103 can be transferred to a holding tank 170 via a pipe 140e. In some other embodiments, the pipe 140e can transfer the treated fluid 103 to a different component. The pipe 140e can also transfer the treated fluid 103 directly to its application to a material. For example, the pipe 140e can transfer the treated fluid 103 to a set of sprinklers that spray the treated fluid 103 onto an arrangement of agricultural items. In some embodiments, a pipe 140f can transfer the treated fluid 103 from the holding tank 170 to its application. In some other embodiments, the holding tank 170 can connect to the pipe 140c to allow the treated fluid 103 to pass through the aspirator 120 as previously described multiple times. This process is referred to as plasma reactivation and has been shown to be effective.

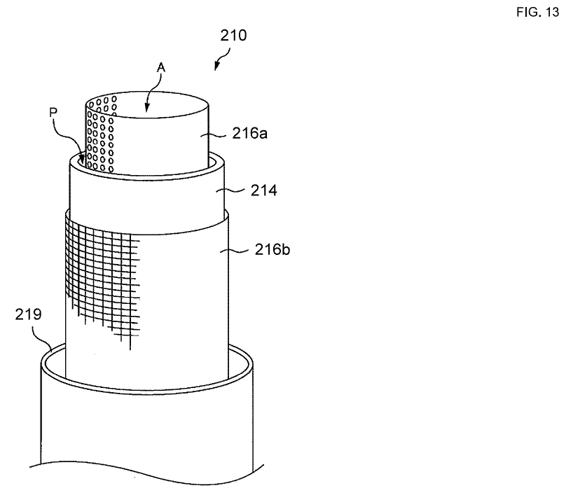

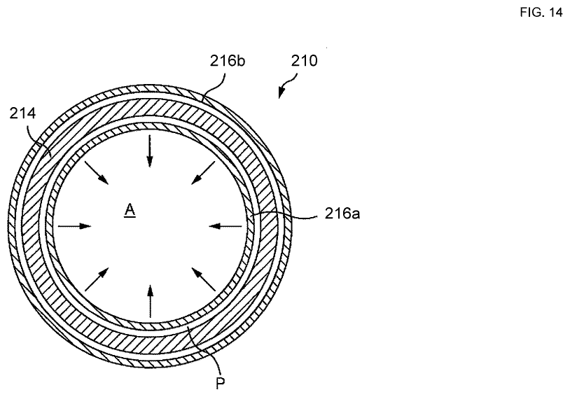

FIG. 13 and FIG. 14 shows an exemplary embodiment of a plasma generator 210 that can be used in the liquid processing apparatus 1 instead of the plasma generator 10 or the plasma generator 110. The plasma generating unit 210 is formed by concentrically combining a plurality of cylindrical members. These cylindrical members are, from the inside, the first electrode 216a, the dielectric layer 214, the second electrode 216b, and the exterior body 219. In FIG. 13, the cylindrical members are shown shifted from each other in the axial direction for easy viewing, but the plasma generating unit 210 is a generally cylindrical device in which cylindrical members of the same length are concentrically arranged. Since the cylindrical dielectric layer 214 is provided between the first electrode 216a and the second electrode 216b so as to be in contact with the second electrode 216b, a dielectric barrier discharge can be induced by applying an AC voltage between the first electrode 216a and the second electrode 216b. The dielectric barrier discharge generates plasma in the gap between the first electrode 216a and the dielectric layer 214. Hereinafter, this gap is referred to as “plasma generation layer P”. It should be noted that the plasma generating unit 210 may be configured such that the cross-sectional shape is not circular. The entire apparatus can be encased by a cylindrical insulation material 219. In some embodiments, there is a gap between the insulation 219 and the second electrode 216b. In some other embodiments, however, there is no gap between the insulation 219 and the second electrode 216b. In some further embodiments, the insulation 219 can be a resin material that fully surrounds the second electrode 219b and acts to both adhere the second electrode 216b to the dielectric layer 214 and to provide insulation to the plasma generator 210.

The first electrode 216a is one electrode for generating plasma, and in some embodiments, is formed as a cylindrical metal tube with a plurality of punched holes. In some embodiments, the first electrode 216a may be formed from a metal mesh material by braiding a thin conducting wire. Good results are yielded when the thickness of the tube is between 0.1 mm and 2 mm with better results between 0.5 mm and 1 mm. The diameter of each punched hole must be large enough to allow efficient flow of gas through the first electrode 216a, but small enough to keep a large surface area of the first electrode 216a. Therefore, good results are yielded when the diameter of the punched holes is between 0.5 mm and 2 mm with better results at 1 mm. Good results are yielded when the distance interval between punched holes is between 1 mm and 10 mm with better results at 5 mm. The material of the first electrode 216a may be any conductive metal, examples of which may include but not be limited to a copper foil or stainless steel. It may be beneficial for the electrodes 216a, 216b to be mirror polished to assist with plasma generation.

For the second electrode 216b, a mesh-like metal material, for example, a metal mesh material formed by braiding a stainless thin wire, can be used. However, embodiments are intended to cover any type of conductive metal mesh material. In some embodiments, the second electrode 216b may be formed as a cylindrical metal tube with a plurality of punched holes. As shown in FIG. 13, a cylindrical dielectric layer 214 is disposed inside the metal mesh cylinder serving as the second electrode 216b. Good results are yielded when the thickness of the dielectric layer 214 is between 1 and 5 mm with better results at 2 mm. A gap is provided between the dielectric layer 214 and the first electrode 216a to form the plasma generation layer P. Good results are yielded when the plasma generation layer P is between 1 and 5 mm with better results at 2 mm. Good results are yielded when the diameter of the second electrode 216b is between 30 mm and 80 mm with better results between 40 mm and 55 mm and even better results at 45 mm. The cylindrical plasma generating unit 210 shown in FIGS. 13 and 14 may have any axial length that in some embodiments is only limited by the power requirements. In some embodiments, there is a linear increase of required power with the axial length of the plasma generating unit 210. In some embodiments, the relationship between required power and axial length may not be linear. In some other embodiments, the relationship between required power and axial length may be linear in some regimes and nonlinear in other regimes. For example, a plasma generator 210 with a certain set of parameters may have a linear relationship between axial length and required power for the first 200 mm, then an exponential relationship for any axial length thereafter. It may therefore be beneficial to select an axial length based on the other parameters of the plasma generator 210 and the power allowance.

The operation of the plasma generator 210 is as follows. First, a gas is supplied to one end of the plasma generator 210. The gas will flow through the plasma generator 210 in the axial direction from the end of the plasma generator 210 it was introduced. The gas will flow through any gaps between the layers of the plasma generator 210, although most of the gas will flow through the plasma generation layer P and the cylindrical space A. When a predetermined AC voltage is applied between the first electrode 216a and the second electrode 216b, an atmospheric low-temperature plasma discharge is generated in the plasma generating layer P between the first electrode 216a and the second electrode 216b, and the active oxygen species including various radicals such as singlet oxygen (1O2), ozone (O3), hydroxyl radical (OH), superoxide anion radical (O2-), hydroperoxy radical (HO2), and hydrogen peroxide (H2O2) are generated by acting on the gas or vapor molecules in the plasma generating layer P. The reactive species can flow through the plasma generating layer P and out of the end of the plasma generator 210 or can flow through the pores in the first electrode 216a and into the cylindrical space A. The gas and reactive species in the plasma generating layer P and the cylindrical space A then flow out of the opposite end of the plasma generator 210 as the end the gas was introduced to.

The plasma generator 210 can be powered by the power supply 20 described in the plasma generator section. The voltage signal would be applied between the first electrode 216a and the second electrode 216b. Good results are yielded when the voltage signal is a sinusoidal wave with a frequency between 30 Hz and 90 Hz. Good results are also yielded when the RMS voltage applied between the electrodes 216a and 216b is between 500 V and 20 kV with better results between 8 kV and 15 kV and even better results at 12 kV.

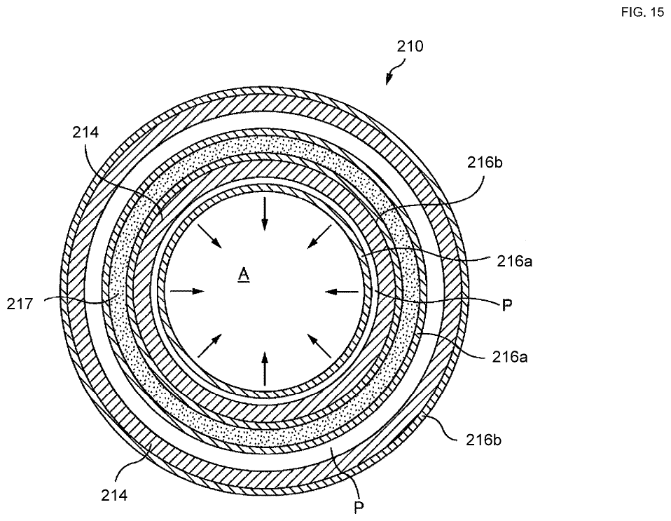

FIG. 15 shows a second exemplary embodiment of the plasma generator 210. This embodiment of the plasma generator 210 includes every component from the exemplary embodiment of the plasma generator 210 described in FIGS. 13 and 14. The embodiment of the plasma generator 210 described in FIG. 15 adds multiple surrounding cylindrical layers of material. The second electrode 216b is surrounded by an insulation gap 217. The insulation gap 217 can be made of a solid material, or can be made of an insulating liquid or gas. In a simple case, air can be used as the insulation gap 217. A supplementary first electrode 216a surrounds the insulation gap 217. In this way, it is apparent that the purpose of the insulation gap 217 is to electrically insulate the second electrode 216b from the supplementary first electrode 216a that surrounds it. A supplementary dielectric layer 214 then surrounds the supplementary first electrode 216a with a gap P between the two. A supplementary second electrode 216b then surrounds the supplementary dielectric layer 214. As in the exemplary embodiment of the plasma generator 210 described in FIGS. 13 and 14, when the supplementary first electrode 216a is connected to the same voltage source as the first electrode 216a and the supplementary second electrode 216b is connected to the same voltage source as the second electrode 216b, a plasma is formed in the plasma generation gap P between the supplementary first electrode 216a and the supplementary dielectric layer 214. By adding the supplementary layers, more plasma can thus be formed. In some embodiments, the supplementary first electrode 216a and the supplementary second electrode 216b are each connected to different voltage sources than the first electrode 216a and the second electrode 216b. The supplementary first electrode 216a, the supplementary dielectric layer, and the supplementary second electrode 216b follow the same dimensional guidelines as the first electrode 216a, the dielectric layer 214, and the second electrode 216b in terms of radial thickness and length, as well as being made of the same or similar materials. The insulating gap 217 can be any thickness that allows proper electrical insulation between the second electrode 216b and the supplementary first electrode 216a, which depends on the material used to form the insulating gap 217. When the material used to form the insulating gap 217 is simply air, good results are yielded when the thickness of the insulating gap is greater than 2 mm and better results are yielded when the thickness of the insulating gap 217 is greater than 3 mm.

The exemplary embodiment of the plasma generator 210 described in FIG. 15 can be modified further to include an n number of supplementary first and second electrodes 216a,216b, supplementary dielectric layers 214, and insulation gaps 217. The number of supplementary layers can be decided based on the power limits and the amount of plasma required for the specific application.

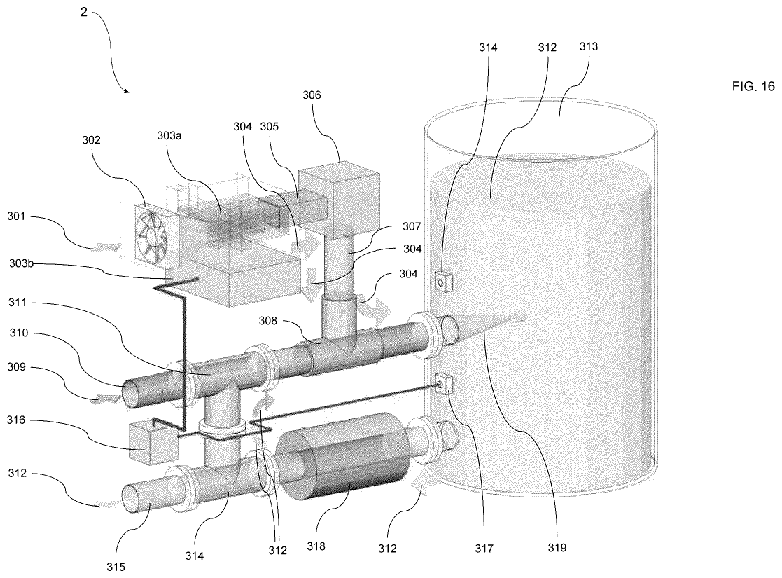

FIG. 16 shows an exemplary embodiment of a liquid processing apparatus 2 that uses much of the same processes and methodology of the liquid processing apparatus 1 from FIG. 8, but is designed to process a larger volume of fluid. Air or other gas 301 enters the apparatus 2 through the air intake member 302 and passes through the plasma generating unit 303a. The plasma generating unit 303a can be the plasma generator 10, the plasma generator 110, or the plasma generator 210, all of which have been described in detail above. The plasma generating unit 303a forms reactive species that are carried by the air 301. The arrows 304 denote the direction of flow of the reactive species within the liquid processing apparatus 2. The air carries the reactive species from the plasma generator through a duct 305 connected to a check valve 306. The check valve 306 can either allow the full flow of air and reactive species, restrict the flow of air and reactive species, or entirely impede the flow of air and reactive species. If the valve 306 allows a flow of air and reactive species, the mixture will continue through a pipe 307 connected to a fluid mixer 308. The fluid mixer 308 is designed to efficiently mix the flow of air and reactive species with a stream of water. In some embodiments, the fluid mixer 308 may implement the same or a similar design as the aspirator 120. Once the reactive species are mixed with a stream of water in the fluid mixer 308, the resulting fluid can be referred to as plasma activated water 312, and is stored in a tank 313. In some embodiments, the plasma activated water 312 is supplied to the tank 313 through an injection nozzle 319. In some embodiments, the injection nozzle 319 can include a babbling head to aid injection.

The stream of water to the fluid mixer 308 can be supplied by a supplemental water injection 309 via a pipe 310 into a fluid mixer 311. The fluid mixer 311 is designed to either allow stream of supplemental water 309 to pass through unchanged, or mix the supplemental stream of water 309 with plasma activated water 312 from the tank 313. The plasma activated water 312 is removed from the tank 313 via a water conveying pump 318, which directs the plasma activated water 312 to a water diversion instrument 314. The water diversion instrument 314 allows a flow of plasma activated water 312 to a pipe 315, the fluid mixer 311, or both the pipe 315 and the fluid mixer 311 at the same time. Once the plasma activated water 312 flows through the pipe 315 it can be supplied to the point of use. In some embodiments, the fluid mixer 311 can allow the plasma activated water 312 to flow to the fluid mixer 308 without mixing any supplemental water 309. In this case, the apparatus 2 would be operating a plasma reactivation scheme, described above.

In some embodiments, the fluid mixers described above may also have the function of a valve, which can be used to allow fluid to flow at a specified rate, or to prevent any flow at all. In some embodiments, the fluid mixers described above may also have the function of only allowing fluid to flow in a specified direction. In some embodiments, the fluid mixers may incorporate active electronic devices, such as a pump or electronic valve, in order to accomplish these functions, or passive devices with no moving parts, such as a fluidic diode.

In some embodiments, an ultrasonic oscillator 314, which may be the same as or similar to the ultrasonic oscillator described in the overview of the plasma activated water section, can be added to the apparatus 2. In FIG. 16, it is shown on the side of the tank 313, but embodiments are intended to cover placement on all other points on the liquid processing apparatus 2. In some embodiments, a concentration sensor 317, which returns the value of the concentration of certain reactive species within the plasma activated water 312, is connected to a drive control device 316, which can modify the device operation based on the value returned by the concentration sensor, among other parameters. In some embodiments, the drive control device (as previously described) 316 may be directly connected to the power supply unit 303b (as previously described). In some embodiments, the power supply unit 303b supplies power to the plasma generating unit 303a. In some other embodiments, the power supply unit 303b not only supplies power to the plasma generating unit 303a, but also to the other components in the apparatus 2 that require power, including but not limited to the concentration sensor 317, the ultrasonic oscillator 314, the check valve 306, the drive control device 316, and the water conveying pump 318.

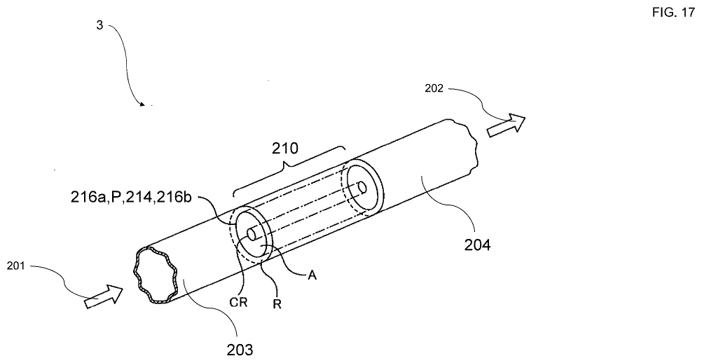

FIG. 17 shows an exemplary embodiment of a liquid processing apparatus 3 that uses the plasma generator 210. First, an untreated liquid 201 is introduced to one side of the liquid processing apparatus 3 through an entry duct 203. The entry duct 203 allows the untreated fluid 201 to flow through one end of the plasma generator 210. The plasma generator 210 treats the untreated fluid 201, transforming it into a treated fluid 202, that can exit the liquid processing apparatus 3 through an exit duct 204.

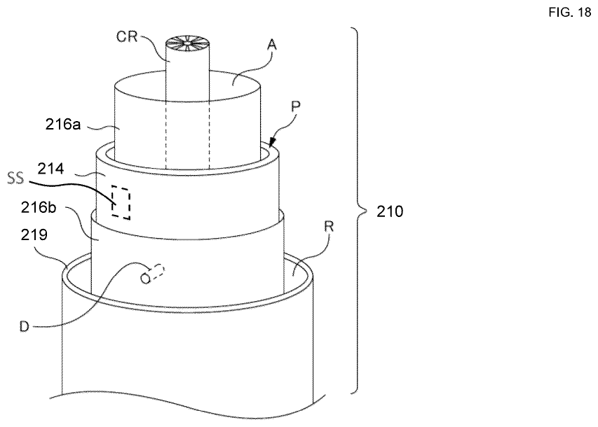

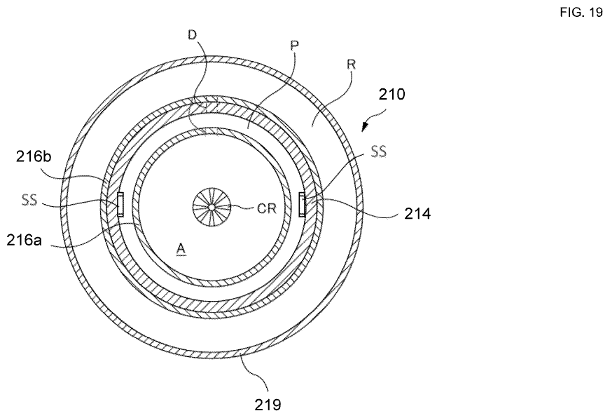

The plasma generator 210 in the liquid processing apparatus is described in FIGS. 18 and 19. The plasma generator 210 is arranged into nested cylindrical layers of material. The plasma generator 210 forms a long pipe shape, and the first electrode 216a, the dielectric layer 214, the second electrode 216b, and the exterior body 219, each of which is a pipe-shaped element, are arranged concentrically from the inside. The first electrode 216a and the second electrode 216b are each formed in a pipe shape by conductive material such as an aluminum plate, stainless steel plate, or similar. Good results are yielded when the first and second electrodes have a thickness between 0.1 and 3 mm, with better results between 0.5 mm and 2 mm and even better results at 1 mm. Good results are also yielded when the dielectric layer 214 has a thickness of between 1 mm and 5 mm with better results between 2 mm and 4 mm and even better results at 3 mm. The outer surface of the dielectric layer 214 is provided in contact with the inner peripheral surface of the second electrode 216b.

The dielectric layer 214 can be formed by selecting an appropriate material in consideration of processability and required dielectric constant, and can be, for example, a glass layer. Good results are yielded when the material of the dielectric layer 214 has a dielectric constant between 2 and 50, with better results between 2 and 10, and even better results at 4.6. The inner peripheral surface of the dielectric layer 214 and the outer peripheral surface of the first electrode 216a face each other, with a gap between the two surfaces defining a plasma generating layer P. Good results are yielded when the plasma generating layer has a thickness between 1 mm and 5 mm, with better results between 2 mm and 4 mm, and even better results at 3 mm. In some embodiments, on the surface of the dielectric layer 14 facing the plasma generating layer P, at least one ultrasonic oscillation element SS is installed at a position facing each other in the radial direction of the plasma generator 210 The ultrasonic oscillator SS may be the same as or similar to the ultrasonic oscillator described in the Overview of the Plasma Activated Liquid section.

FIG. 18 shows an outer insulation layer 219 is provided outside the second electrode 216b at a distance that defines a gap between these two called the annular space R. A circulator CR is provided at the center of the plasma generator 210 for exerting a stirring action in the cylindrical space A inside the inner radius of the first electrode 216a. The circulator CR takes the form of a cylindrical member extending along the axis of the plasma generator 210. In some embodiments, the circulator CR includes a rotating blade for agitating operation and a driving mechanism for rotating and driving the rotating blade. However, the configuration of the circulator CR is not limited to a specific configuration and embodiments are intended to include any structure with the function of stirring the fluid in the cylindrical space A. In some embodiments, the circulator CR includes a power supply and control unit, as previously described (not pictured).

An elongated hollow duct D is provided between the annular space R and the plasma generating layer P so as to penetrate through the second electrode 216b, the dielectric layer 214, and the first electrode 216a. In some embodiments, the duct D is formed of a metal material such as a thin stainless tube, although embodiments are intended to cover any material that can provide a passage for a gas. In some embodiments, the duct D is merely a hole in the second electrode 216b and the dielectric layer 214 such that a gas can flow through. The duct D can also continue to penetrate through the first electrode 216a, thus providing a passage for gas from the plasma generating layer P to the cylindrical space A. In some embodiments, the duct D from the annular space R to the plasma generating layer P can be radially positioned over the duct D from the plasma generating layer P to the cylindrical space A. In some other embodiments, the duct D from the annular space R to the plasma generating layer P can be positioned in a different angular location and thus not directly over the duct D from the plasma generating layer P to the cylindrical space A. Although only one duct D is shown in FIGS. 18 and 19, an appropriate number of ducts D can be provided at appropriate intervals along the axial direction of the plasma generator 210.

FIG. 17 shows the untreated liquid 201 being introduced into the plasma generator 210 from the entry duct 203, the untreated liquid 201 is introduced into the cylindrical space A and is not introduced into the plasma generating layer P or the annular space R. In some embodiments, the diameter of the pipe 203 is the same as the diameter of the cylindrical space A in order to prevent the untreated liquid 201 from flowing into the plasma generating layer P or the annular space R. Then a gas such as outside air is first introduced into the annular space R and enters the plasma generation layer P through the duct D to be subjected to plasma treatment. As discussed above, the plasma generating layer P is the region where reactive species are formed and are suspended in the gas. The gas that now contains reactive species is then introduced through the duct D from the plasma generating layer P and into the liquid in the cylindrical space A, where it is mixed with the untreated liquid 201. The mixing can be accomplished by the circulator CR so that untreated liquid 201 is quickly and efficiently mixed with the gas containing reactive species to become a treated liquid 202.

What is claimed is:

1. A plasma generator apparatus for generating atmospheric pressure, low temperature plasma that can communicate with a gas to generate reactive species that can be contained in a liquid, the plasma generator comprising:
a first electrode that defines a bottom surface, the first electrode having a width and length that are each greater than a height extending in a height direction that extends at an angle relative to the bottom surface;
a second electrode that defines a top surface, the second electrode having a width and length that are each greater than a height extending in the height direction that extends at an angle relative to the top surface, the second electrode opposing the first electrode such that the bottom surface of the first electrode faces and is separated from the top surface of the second electrode by a space that is configured to house the gas;
a dielectric layer that defines a surface that is disposed in at least a part of the space between the bottom surface of the first electrode and the top surface of the second electrode, the dielectric layer having a relative permittivity between 2 and 500, and a thickness of 3 mm or less;
a power supply configured to supply electrical power to the first and second electrodes at a predetermined voltage and frequency, such that, based on a distance between the first and second electrodes, and the presence of the dielectric layer, atmospheric pressure, low temperature plasma is generated in the space so as to communicate with the gas disposed therein to thereby generate the reactive species;
a duct disposed to communicate with the space and configured to accept the generated reactive species; and
a channel configured to house the liquid, the channel disposed to communicate with the duct to enable the reactive species to become contained in the liquid.

2. The plasma generator of claim 1, wherein the power supply is configured to supply AC electrical power, and to be adjustable to provide desired AC voltages to generate stable atmospheric pressure, low temperature plasma.

3. The plasma generator of claim 1, wherein the power supply includes an inverter that is configured to converts DC voltage to AC voltage and is configured to output AC20V-AC100V.

4. The plasma generator of claim 3, wherein the inverter is configured to output AC25V-AC45V.

5. The plasma generator of claim 2, wherein the inverter is configured to output an applied voltage with a frequency ranging from 30 Hz-90 Hz.

6. The plasma generator of claim 2, wherein the power supply includes a booster that receives the output of the inverter and boosts the received voltage at a rate of 150× at 2× intervals, ranging from 500V-20 kV.

7. The plasma generator of claim 1, wherein the surface of the dielectric layer forms a cylinder, having a length that is the axial distance of the cylinder, a width that is a circumference of the cylinder, and a height that is the difference between an outer radius and an inner radius of the cylinder.

8. The plasma generator of claim 7, wherein the first and second electrodes are formed into strips with a length greater than the width and arranged into a double helix structure around the dielectric surface, the double helix structure of the first and second electrodes defining a helix diameter that equals the outer radius of the dielectric surface and a helix angle.

9. The plasma generator of claim 8, wherein the width of the dielectric surface divided by pi is between 2 mm and 20 mm.

10. The plasma generator of claim 9, wherein the width of the dielectric surface divided by pi is between 3 mm and 10 mm.

11. The plasma generator of claim 10, wherein the width of the dielectric surface divided by pi is approximately 4 mm.

12. The plasma generator of claim 9, wherein the height of the dielectric surface is between 0.1 mm and 2.5 mm.

13. The plasma generator of claim 9, wherein the thickness of the first and second electrodes is between 0.01 mm and 0.1 mm.

14. The plasma generator of claim 13, wherein the thickness of the first and second electrodes is approximately 0.05 mm.

15. The plasma generator of claim 8, wherein the helix angle of the first and second electrodes is between 30 degrees and 75 degrees.

16. The plasma generator of claim 15, wherein the helix angle of the first and second electrodes is between 50 degrees and 70 degrees.

17. The plasma generator of claim 16, wherein the helix angle of the first and second electrodes is approximately 64.5 degrees.

18. The plasma generator of claim 7, wherein the first and second electrodes are formed into cylinders where the length is the axial distance of each cylinder, the width is the circumference of each cylinder, and the height is the difference between the outer radius and the inner radius of each cylinder.

19. The plasma generator of claim 18, wherein the inner radius of the first electrode is greater than the outer radius of the dielectric layer and the outer radius of the second electrode is less than the inner radius of the dielectric layer.

20. The plasma generator of claim 1, wherein the dielectric layer includes a first dielectric layer and a second dielectric layer, the first and second electrodes and the dielectric layers forming planar surfaces.

21. The plasma generator of claim 20, wherein the first dielectric layer is disposed on the planar bottom surface of the first electrode, and the second dielectric layer is disposed on the planar top surface of the second electrode.

22. The plasma generator of claim 21, wherein, for each of the first and second dielectric layers, the relative permittivity is between 2 and 15, and thickness is between 1 mm and 3 mm.

23. The plasma generator of claim 21, wherein, for each of the first and second dielectric layers, the relative permittivity is between 15 and 100, and thickness is less than 2 mm.

24. The plasma generator of claim 21, The plasma generator of claim 1, wherein, for each of the first and second dielectric layers, the relative permittivity is between 100 and 500, and thickness is less than 1 mm.

25. The plasma generator of claim 1, further comprising a plasma generation region where reactive oxygen species and reactive nitrogen species are generated and ejected.

26. The plasma generator of claim 1, further comprising an aspirator configured to efficiently mix the ejected reactive species with a jet of fluid.
